(19) United States
(12) Patent Application Publication
(10) Pub. No.: US 2025/0314652 A1
(43) Pub. Date: Oct. 9, 2025

(54) IMMUNOASSAY FOR DETECTING BIOLOGICALLY ACTIVE PROTEINS

(71) Applicant: PHAEOSYNT GMBH, Hannover (DE)

(72) Inventors: Alina EILERS, Hannover (DE); Eva-Maria PLOENNIGS, Wennigsen (DE); Stephanie PFEIL-COENEN, Lindwedel (DE)

(21) Appl. No.: 19/111,956

(22) PCT Filed: Sep. 15, 2023
(86) PCT No.: PCT/EP2023/075545

§ 371 Date: Mar. 14, 2025

(30) Foreign Application Priority Data
Sep. 15, 2022 (LU) LU103006

(51) Int. Cl. G01N 33/543; G01N 33/76

(52) U.S. Cl. CPC G01N 33/54388; G01N 33/76; G01N 2333/075; G01N 2333/11; G01N 2333/16; G01N 2333/165; G01N 2333/315; G01N 2333/405; G01N 2333/5421; G01N 2333/57; G01N 2333/59; G01N 2333/70539

(57) ABSTRACT

The present invention relates to a vegan immunoassay for detecting a biologically active antigen, a device in which such an immunoassay is arranged, a kit comprising such a device as well as the use of the immunoassay and a method for detecting a biologically active antigen.

### TECHNICAL FIELD

The present invention relates to an immunoassay for detecting a biologically active protein, a device in which such an immunoassay is arranged, a kit with such a device, the use of the immunoassay and a method for detecting a biologically active antigen.

### STATE OF THE ART

There are many different immunoassays for analyzing body fluids from an individual, such as whole blood, serum, saliva, milk or urine.

One problem with common immunoassays, which make use of antibodies, consists in the fact that the antibodies are of animal origin. Thus, antibodies are produced either in animals or in animal cell cultures. In particular, CHO— (oviduct cells of the Chinese hamster) and HEK-293T cells (human embryonic kidney cells) are employed. This cell culture-based production is very cost-intensive; therefore, available capacities are primarily employed in the production of high-priced, therapeutic antibodies. In the field of diagnostics, antibodies exhibit much lower margins and are consequently still frequently produced in animals. Such antibodies produced in animals possess not only the associated animal suffering but also the low reliability of binding properties, limited availability, and the risk of contamination with human pathogens. To date, no technical solutions exist that would allow the manufacture of purely vegan assays in a cost-effective manner and in economically relevant production volumes.

The alternatives for producing antibodies, as well as other complex proteins, are quite limited. Bacteria such as Escherichia coli cannot carry out the necessary posttranslational modifications on the antibodies, furthermore, the production rates are extremely low. A similar situation applies to yeast- or Pichia-based production systems. Here, too, the antibodies can rarely be folded correctly by the host cells and the production rates are similarly low as in bacteria. Transgenic plants have been discussed for many years as alternative producers, but even here the very low production rates do not permit economically viable exploitation.

Attempts have been made to use higher plants for the production of antibodies, as disclosed, for example, in patent document U.S. Pat. No. 6,080,560 A. Additional approaches are disclosed in publications, for example by Melnik Stanislav et al. (DOI: 10.1111/pbi.12746) and by Ayala Marta et al. (DOI: 10.1007/978-1-59745-407-0_7). The problem with these plants is that the production rates of antibodies in plants are generally very low, since typically only the cells of some tissues of the plant have integrated the antibody genes into their genome, whereby only certain tissues produce small amounts of antibodies. The plant is a chimera, as it contains many tissues that have not incorporated any antibody genes, which further reduces the yield. In order for an antibody to be produced over several generations, it must additionally be ensured that the germline cells have integrated the antibody genes into their genome, in order to guarantee permanent production. Finally, permanent production requires selfing or grafting of the transgenic plants. With a “natural” sexual reproduction, the transgene would no longer be present after only a few generations.

An alternative method uses the agroinfiltration of genetically modified plant viruses, which are used to infect plants. A problem with these plants is that they are transiently modified, i.e. the antibody genes are not integrated into any of the plant genomes, so that they can only stably express antibodies over a single generation. In this approach, every cell of the plant is indeed capable of producing the antibodies, since the virus disseminates throughout the plant. However, in this case there is no integration into the plant genome and the plant does not survive this procedure, but dies within a few days. This means that a consistently stable production cannot be ensured, and legally it always involves the regeneration of transgenic plants with all the corresponding regulatory implications.

Furthermore, purification is problematic as higher plants produce significant amounts of fibrous material, which makes it difficult to separate and isolate antibodies and is therefore not suitable, to provide antibodies of comparable quality and quantity to the animal alternatives.

Furthermore, there are already several known research documents and patent specifications for the expression of antibodies from diatoms. Examples in the literature of secreting antibodies from diatoms include the work published by Hempel et al. (DOI: 10.1186/14 75-2859-11-126), Samuels et al. (DOI: 10.1038/s41598-022-11053-7) and Hempel et al. (DOI: 10.1371/JOURNAL.PONE.0028424). A shared feature of these approaches is that these antibodies are expressed extracellularly, which has the unfortunate disadvantage that these methods are not capable of producing homogeneous, uniform antibodies of high quality and in technically feasible quantities that can be used commercially as an alternative to established antibodies from animal sources. This is also the case with the following patent specifications. The production of antibodies in Phaeodactylum tricornutum, a microalgae, was described. For example, EP 2 671 950 A1 discloses the expression and secretion of recombinant, fully assembled protein complexes by microalgae. Here, the microalgae are expressed extracellularly, which theoretically leads to easier separation of the proteins; an unfortunate disadvantage of this method is the very low production rates, which means that the antibodies can unfortunately only be purified at very high expense. The patent specifications EP 2 444 495 A1 and EP 2 660 323 A1 describe an approach for the production of therapeutic antibodies using a transformed microalgae called Phaeodactylum tricornutum. The transformed microalga contains a nucleic acid sequence that encodes for a therapeutic antibody, a functional fragment or a derivative thereof, coupled with a heterologous signal peptide, all operably linked with a promoter. These transformed microalgae express the therapeutic antibodies extracellularly into the medium, i.e. the antibodies are secreted. This approach, unfortunately, yields only small amounts of therapeutic antibodies, which is attributable to the inefficient use of extracellular expression.

### Objective

The technical objective underlying the present invention is therefore to provide an immunoassay which overcomes the disadvantages known from the prior art, in particular the provision of recombinant antibodies from a diatom or unicellular plant in high quality and technically usable quantities.

### Solution

To solve the technical problem of the prior art, a preferred embodiment of the present invention for detecting a biologically active antigen, in particular a hormone, protein or vaccine, in a biological sample of an individual comprises the use of recombinant antibodies e.g. a first antibody, a second antibody and/or a further antibody, which are obtained from a diatom, a unicellular plant or Viridiplantae, wherein the recombinant antibody comprises a heterologous diatom-, plant- or microalga-specific signal peptide and/or a glycosylation pattern that deviates from that of a native antibody obtained from an individual (as defined herein).

According to a preferred embodiment of the present invention, at least two antibodies, for example the first antibody and the second antibody, particularly preferably all antibodies used were obtained from a diatom, unicellular plant or Viridiplantae.

The objective is solved by an immunoassay with the features of claim 1 and a device and a method with the features of the dependent claims. Further advantageous embodiments can be found in the subclaims, the description and the embodiment examples. The advantages of the immunoassay and the other components are shown below in the further description.

According to a preferably embodiment of the present invention there is provided an immunoassay for detecting a biologically active antigen, particularly a hormone, protein or vaccine in a biological sample of an individual, comprising the following components:
a sample application area for applying a biological sample of an individual, wherein the biological sample is preferably urine, whole blood, saliva, milk or serum of an individual;
a capture area, wherein the capture area is provided with an immobilized first antibody that is directed against the biologically active antigen, in particular against the hormone, the protein, the protein or the vaccine;
a conjugate area, wherein the conjugate area is provided with a second antibody directed against the biologically active antigen, particularly against the hormone, the protein, the peptide or the vaccine; and optionally
a control region which provides an antibody that is directed against the detection antibody;

wherein the first antibody and/or the second antibody is a recombinant antibody obtained from a diatom, unicellular plant or Viridiplantae, particularly obtained by a method as defined herein,

wherein the recombinant antibody has a heterologous diatom-, plant- or microalga-specific signal peptide and/or a glycosylation pattern different from that of a native antibody obtained from an individual (as defined herein).

Particularly preferred is the problem solved by an immunoassay for detecting a biologically active antigen, particularly a hormone, protein or pharmaceutical substance in a biological sample of an individual, provided with:
a sample application area for applying a biological sample of an individual, wherein the biological sample is preferably urine, whole blood, saliva, milk or serum;
a capture area, wherein the capture area is provided with an immobilized first antibody that is directed against the biologically active antigen, in particular against the hormone, the protein, the peptide or the pharmaceutical substance;
a conjugate area, wherein the conjugate area is provided with a second antibody directed against the biologically active antigen, particularly against the hormone, protein, peptide or pharmaceutical substance;

wherein the first antibody and/or the second antibody is a recombinant antibody obtained from a diatom or unicellular plant,

wherein the recombinant antibody is provided in a purity of at least 90%, preferably at least 95%. In an alternatively preferred embodiment, the recombinant antibody is provided in a purity of 80 to 99% purity, more preferably 85 to 99%, more preferably 90 to 99%, most preferably 95 to 99%.

The biological sample of an individual may be whole blood, serum, saliva, urine or milk. Preferably, the biological sample of the individual is urine, whole blood or serum, particularly preferred urine.

According to a preferably embodiment of the present invention, the immunoassay is a lateral flow immunoassay. For example, the lateral flow immunoassay provides a sample application area, a conjugate area and a capture area arranged on a membrane, wherein a sample application area and a capture area on the membrane are fluid-connected to each other via a flow path, and wherein a conjugate area is arranged in the flow path. For example, the membrane is a nitrocellulose membrane.

In a preferably embodiment, the immunoassay provides a nitrocellulose membrane.

Alternatively preferably, the immunoassay is an enzyme-linked immunosorbent assay (ELISA), e.g. a direct ELISA, an indirect ELISA, a direct sandwich ELISA or an indirect sandwich ELISA, preferably the ELISA is a direct sandwich ELISA or an indirect sandwich ELISA.

It may be expedient for the first antibody and/or the second antibody, in particular the mobilized one of the two antibodies, to be labeled with a dye and/or an optically active nanoparticle, in particular a gold nanoparticle.

Nevertheless, it may be provided that the first antibody and/or the second antibody, particularly the mobilized one of the two antibodies, is coupled with an enzyme which is adapted to induce a dye or a luminescence reaction

### General Advantages

For the provision of the immunoassay or a method for detecting a biologically active antigen in a biological sample of an individual, recombinant antibodies are used which are not of mammary synthesis origin, so that advantageously animals or animal cell cultures, particularly mammary cell cultures, can be dispensed with.

Furthermore the synthesis/expression of the recombinant antibodies used herein not only enables similarly high expression rates to be achieved in a diatom with much lower energy and resource requirements, but also eliminates the risk of the antibodies being contaminated with human pathogens, as is the case with synthesis/expression in animals or animal cell cultures.

### DETAILED DESCRIPTION OF THE INVENTION

In an embodiment of the present invention, an immunoassay for detecting a biologically active antigen, particularly a hormone, protein or pharmaceutical substance in a biological sample of an individual is provided with:
a sample application area for applying a biological sample of an individual, wherein the biological sample is preferably urine, whole blood, saliva, milk or serum;
a capture area, wherein the capture area is provided with an immobilized first antibody that is directed against the biologically active antigen, in particular against the hormone, the protein, the peptide or the pharmaceutical substance;
a conjugate area, wherein the conjugate area is provided with a second antibody directed against the biologically active antigen, particularly against the hormone, protein, peptide or pharmaceutical substance;

wherein the first antibody and/or the second antibody is a recombinant antibody obtained from a diatom, unicellular plant or Viridiplantae, wherein the recombinant antibody comprises a heterologous diatom-, plant- or microalga-specific signal peptide.

In a particularly preferred embodiment of the present invention, an immunoassay for detecting a biologically active antigen is provided, particularly for detecting a hormone, protein or pharmaceutical substance in a biological sample of an individual, providing:
a sample application area for applying a biological sample of an individual, wherein the biological sample is preferably urine, whole blood, saliva, milk or serum;
a capture area, wherein the capture area is provided with an immobilized first antibody that is directed against the biologically active antigen, in particular against the hormone, the protein, the peptide or the pharmaceutical substance;
a conjugate area, wherein the conjugate area is provided with a second antibody directed against the biologically active antigen, particularly against the hormone, protein, peptide or pharmaceutical substance;

wherein the first antibody and/or the second antibody is a recombinant antibody obtained from a diatom or unicellular plant, wherein the recombinant antibody is provided in a purity of at least 90%, preferably at least 95%. In an alternatively preferred embodiment, the recombinant antibody is provided in a purity of 80 to 99% purity, more preferably 85 to 99%, more preferably 90 to 99%, most preferably 95 to 99%.

Here, a distinction can be made between two different types of contamination:
Contamination by non-proteins, for example pigments;
Contamination by other proteins that are not antibodies.

Both groups of impurities can interfere with the purification and function of the antibodies by adversely affecting the stability of the antibodies, but also by reducing the specificity of the immunoassays.

Non-proteins can generally be residues from the separation process, particularly cell parts, components of the culture medium and, for example, fibers in higher plants. In the production of antibodies in diatoms, this group mainly includes pigments that can be detected spectrophotometrically and easily separated, as they have a much lower molecular weight than the antibodies and have different chemical properties. For this, common methods known to the specialist such as dialysis, ultrafiltration, size exclusion chromatography or charge-dependent separation (e.g. ion exchange chromatography) are suitable. This separation is coupled with an effort that increases in relation to the amount of isolated antibody, the lower the proportion of antibody in the culture. After separation the amount of non-proteins can preferably be determined gravimetrically, in the case of pigments particularly preferred spectroscopically.

Contamination by proteins, which are not antibodies, can be analyzed by denaturing SDS-polyacrylamide gel electrophoresis (SDS-PAGE), combined with coomassie staining on the one hand, and immunostaining to identify antibodies, preferably antibody chains produced in diatoms, on the other hand. For this purpose, the protein mixtures isolated from diatoms are separated according to size after the addition of an appropriate buffer and denaturation (10 min at 80° C.). This standard procedure in molecular biology is well known to experts (e.g. Reinard, Molekularbiologische Methoden 2.0 (UTB, p. 229 ff; ISBN 978-3825287955). The proteins separated in the SDS-PAGE are stained with Coomassie and the color intensity is measured densitometrically and/or compared with a standard. This makes it possible to determine how much protein is present in each band.

In parallel, a further SDS-PAGE can be carried out with identical application of the samples, which are not stained but transferred to a nitrocellulose membrane. The bands caused by the two chains of the antibody are clearly visible on this membrane. Through the addition of a first antibody specifically directed against the antibody chains, which is labeled, for example with a biotin, radioisotope, reporter enzyme, oligonucleotide or fluorophore, or the addition of a second, labeled antibody, which is directed against the first antibody, the two chains of the antibody to be detected become clearly visible. All bands that are not labeled in this way are contaminating proteins.

In a preferably embodiment, the ratio of recombinant antibody to total protein is determined by SDS-PAGE after separation and spectroscopic quantification of the non-proteins. This method makes it possible to determine the absolute amount of antibodies, total proteins and non-proteins of an obtained recombinant antibody according to the present invention.

The purity of the purified recombinant antibody provided for an immunoassay according to the present invention is calculated according to the following formula:

$$purity ( \% ) = \frac{( Amount of recombinant antibody )}{( Amount of total proteins ) + ( Amount of non - proteins )} \times 100 \%$$

The purity of the recombinant antibody is determined at the time after purification, before the antibody is applied to an immunoassay according to the present invention or is otherwise provided.

In an alternatively embodiment of the present invention, the recombinant antibody is provided in a protein purity of at least 90%, preferably at least 95%. In an alternatively preferred embodiment, the recombinant antibody is provided in a protein purity of 80 to 99% purity, more preferably 85 to 99%, more preferably 90 to 99%, most preferably 95 to 99%.

The protein purity of the recombinant antibody is calculated according to the following formula:

$$Protein purity ( \% ) = \frac{( Amount of recombinant antibody )}{( Amount of total proteins )} \times 100 \%$$

According to a preferably embodiment, the ratio of recombinant antibody to total protein is determined by SDS-PAGE. The protein purity of the recombinant antibody is determined at the time after purification, before the antibody is applied to an immunoassay according to the present invention or otherwise made available.

A particularly preferred embodiment is one in which, due to the high production rate of the recombinant antibody according to the invention, it is obtained in a high concentration of preferably 20-1000 mg/L culture. Here, cell debris and diatom-specific proteins can be easily separated, as interfering fibers are not present. Thereby, a recombinant antibody according to the invention is provided in a purity of at least 90%, preferably of at least 95%. Due to the high purity of the antibody, together with the high homogeneity and uniformity of the glycosylation pattern according to the invention, an antigen can be detected specifically and at a low concentration, i.e. with a low detection limit of the immunoassay, thus achieving an improvement over the previously used immunoassays based on animal antibodies. Due to the higher homogeneity of the antibodies, the number of non-specific cross-reactions (non-specific signals) is lower, making the result of the immunoassay according to the present invention more reliable, wherein the sensitivity of the antibody is consistently high.

Due to the higher purity of the antibodies, they are more stable and have a longer shelf life because antibody-degrading proteases are also removed along with the foreign proteins. Furthermore, the purity and homogeneity of the antibodies mean that the number of non-specific cross-reactions (non-specific signals) is lower, which leads to a more reliable and reproducible result.

In a particularly preferred embodiment, which is shown in FIG. 13, the commercially purchased antibody clearly shows the additional bands due to non-specific reactivity. These are missing on the gel lanes of the antibody produced in diatoms, which shows that it has a higher specificity than the commercial animal antibody. In addition to reducing false-positive signals, this also has the advantage that a smaller amount of protein is required for the immunoassay.

An “immunoassay” is a test that uses the binding of antibodies to antigens to identify specific substances and/or quantify the amount of the substance present. Immunoassays can be used to diagnose diseases, but also to analyze the physiological state of an individual, e.g. human individuals. Examples of disease diagnoses are the detection of various cancers, the detection of an infection such as Covid19. Examples of the analysis of a physiological condition are female conception (ovulation tests) or pregnancy tests. Immunoassay and immunassay are used interchangeably.

Immunoassays can be used in different technical variants. Best-known technical variants are the enzyme-linked immunosorbent assay (ELISA or ELISA test) and the lateral flow immunoassay (LFA), wherein a membrane, e.g. a paper-based platform, is used for detecting and quantifying analytes in complex mixtures, wherein the biological sample is placed on a test device and the results are displayed within 5-30 minutes. Well-known examples of antibody-based LFAs are the Covid-19 rapid tests or pregnancy tests.

The term “biologically active antigen” (also referred to as “antigen”) as used in the present invention refers to any substance, particularly molecules with a molecular weight of about 4,000 Daltons or more, preferably about 2,000 Daltons or more, most preferably 770 Daltons or more, which causes the body to trigger an immune response against that substance. Antigens include toxins, chemicals, bacteria, viruses, proteins, peptides or other substances that come from outside the body. Antibodies are produced during the humoral immune response of vertebrates. These antibodies formed for the humoral immune response can also be used independently of an immune response and outside the body to recognize, identify and/or quantify the antigen. Biologically active antigen and biologically active protein may be used interchangeably for the purposes of the present invention.

From a biological point of view, biologically active antigens are understood as molecules against which an antibody recognizing them exists in an individual (as defined herein). In particular, this refers to molecules that represent proteins and/or hormones from humans and mammals which act either as markers for a disease in the individual (for example, diagnosis of breast cancer by detecting the Herceptin2 receptor) or as markers for the physiological state of the individual (for example, pregnancy). Alternatively preferably, the term refers to molecules that represent proteins and/or hormones in animals and/or plants, particularly for food analysis. Biologically active antigens, particularly sequences of hormones, proteins or vaccines or pharmaceutical substances, particularly preferred hormones, which act either as markers for a disease of the individual (for example diagnosis of breast cancer via the detection of Herceptin2 receptor) or as markers for the physiological state of the individual (for example pregnancy), are known to the person skilled in the art or can be taken from relevant databases and specialist books.

Moreover, biologically active antigens are understood to be proteins from human pathogenic organisms, particularly bacteria (e.g. Streptococcus mutans (these cause tooth decay) or viruses (e.g. spike protein from Covid 19).

Preferably, the biologically active antigen is a hormone of an individual, e.g. a human hormone selected from the group comprising cortisol, thyroxine, somatotropin, vasopressin, testosterone, estrogens. Corresponding native antibodies for detecting the hormones are known to the person skilled in the art or can be found in the relevant literature.

According to a preferably embodiment, the biologically active antigen is a peptide hormone of an individual, e.g. a human peptide hormone selected from the group comprising gonadoliberin (10 amino acids), insulin (A chain: 21 amino acids, B chain: 30 amino acids), somatostatin (14 amino acids), glucagon (29 amino acids). Corresponding native antibodies for detecting the hormones are known to the person skilled in the art or can be found in the relevant literature.

According to a preferably embodiment, the biologically active antigen is a protein hormone of an individual, e.g. a human protein hormone, which serve, for example, as physiological markers. Suitable protein hormones are, for example, selected from the group comprising parathyroid hormone (84 amino acids) HCG (human chorionic gonadotropin (hCG): α-subunit: 92 amino acids, β-subunit: 145 amino acids). Corresponding native antibodies for detecting the hormones are known to the person skilled in the art or can be found in the relevant literature.

Furthermore, the biologically active antigen may be a protein, particularly a human protein, which serves as a disease marker and is selected from the group comprising Her2 (human epidermal growth receptor 2, which is overexpressed on cancer cell surfaces and is a cancer marker); IgE antibodies (which are produced by the human body due to an allergic reaction); small signaling proteins, such as interleukin 5 (which is associated with allergic diseases, including allergic rhinitis and asthma); interferons (which have immunostimulatory, especially antiviral and antitumor effects and can serve as markers for viral infections). Corresponding native antibodies for detecting the hormones are known to the person skilled in the art or can be found in the relevant literature.

The biologically active antigen can be a protein of a human pathogenic organism, e.g. the spike protein of a coronavirus, such as SARS-COV-1 (for detecting SARS infections) or SARS-COV-2 (for detecting a Covid-19 infection) or the HIV-1 nucleocapsid protein (for detecting an HIV infection).

According to a preferably embodiment, the biologically active antigen is a hormone, a protein and/or a pharmaceutical substance. Particularly preferred the biologically active antigen is a hormone, in particular a hormone which acts as a marker for the physiological state of the individual (for example pregnancy), especially preferably chorionic gonadotropin, particularly preferably human chorionic gonadotropin.

In a lateral flow immunoassay (herein also referred to as “LFA”), three antibodies are typically used:
an immobilized first antibody, which is arranged in the capture area (also referred to as the test zone) and is also referred to as the capture antibody. Like the second antibody (also known as the detection antibody), this is also directed against the antigen, preferably against a different epitope of the antigen than the detection antibody;
a second antibody, which is preferably provided in the conjugate area and is also referred to as a detection antibody, which is directed against an epitope of the antigen, e.g. a hormone, a protein or a peptide, such as human chorionic gonadotropin, wherein the second antibody is preferably conjugated to marker particles, e.g. gold, silver, latex, carbon, nanoparticles, fluorescent dyes or enzymes.
a further antibody which is preferably located in a control area (control zone), preferably immobilized, and is directed against a detection-/control antibody.

Preferably, the first antibody (e.g. the capture antibody), the second antibody (e.g. the detection antibody) and/or the further antibody (e.g. the capture antibody in the control zone), particularly preferably all antibodies are obtained from a diatom, unicellular plant or viridiplantae and are characterized by the characteristics defined herein.

Particularly preferably, the first antibody (e.g. the capture antibody), the second antibody (e.g. the detection antibody) and/or the further antibody (e.g. the capture antibody in the control zone), particularly preferably all antibodies, are obtained from a diatom and are characterized by the characteristics defined herein.

An advantage over the use of animal antibodies is also the consistent quality and reproducibility of the antibodies used in the test and thus an improved quality of the entire immunoassay, since, for example, the antibody-producing animals die after a certain time and antibodies from another animal have different properties, i.e. the quality of the antibodies produced in the conventional way fluctuates greatly. Furthermore antibodies and auxiliary proteins obtained from a diatom, unicellular plant or viridiplantae, as opposed to polyclonal antibodies obtained from individuals (as defined herein), are precisely defined antibodies, i.e. their amino acid sequence is predefined and consistent.

The term “individual” (also referred to herein as “subject”), as used in the present invention, means any mammal (e.g. mouse, rat, rabbit, dog, cat, cattle, pig, sheep, horse or primate), particularly rodents, ungulates, hoofed animals, preferably with odd toes, or primates. In a particularly preferred embodiment, the individual is a primate, particularly a human. Unless otherwise specified, the term “individual” does not denote a specific age and therefore comprises adults, older individuals, children and newborns.

In a preferably embodiment of the immunoassay, the individual is a mammal, preferably a primate, more preferably a human.

According to a preferably embodiment of the invention, the recombinant antibodies, for example the first antibody and/or the second antibody and/or the further antibody are obtained from the diatom (also diatoms), the green alga (also Chlorobionta) or a seed plant by expression. Particularly preferred are the recombinant antibodies obtained from the diatom or the green alga, particularly in the diatom, such as Phaeodactylum tricornutum. One advantage of this is that antibodies from plants, particularly from diatoms, cannot contain endogenous pathogens. One example of this is mad cow disease (BSE), which is why animal antibodies can pose a risk, particularly for therapeutic applications. Therefore, these must be strictly tested for possible human pathogens, which is not necessary for the recombinant antibodies according to the invention, particularly preferably obtained from diatoms.

The term “antibody” as used herein means an immunoglobulin (Ig) or a derivative of an immunoglobulin as produced by the acquired immune system of vertebrates. Examples of naturally occurring antibodies are the antibodies of class M (IgM), G (IgG), A (IgA), and E (IgE), particularly from mammals such as humans, rabbits, mice, rats, camels, llamas, goats and/or horses. Furthermore, artificial formats based on such proteins are also included, for example scFvs or scFv-Fc.

In some preferably embodiments of the immunoassay, “antibody” herein refers to an immunoglobulin (Ig) or a derivative of an immunoglobulin such as that produced by the acquired immune system of vertebrates and/or cartilaginous fish. Examples of naturally occurring antibodies are the antibodies of class M (IgM), D, (IgD), G (IgG), A (IgA), and E (IgE), NAR (IgNAR), particularly from mammals such as humans, rabbits, mice, rats, camels, llamas, goats and/or horses and/or cartilaginous fish such as sharks. Furthermore, artificial formats based on such proteins are also included, examples of which are scFvs, scFv-Fc or single-domain antibodies/nanobodies.

A “native antibody” in the context of the present invention is a natural antibody as it is found in an individual as defined herein, in particular a vertebrate, particularly preferred a mammal, most preferably a primate, in particular a human.

According to a preferably embodiment, the antibody comprises at least a variable region and a constant/preserved region.

Preferably, the variable region is of vertebral, preferably mammary origin, particularly preferably of human, murine, equine, canine and/or camel origin, very particularly preferably of human, murine, equine and/or camel origin, particularly of human, murine and/or equine origin. The variable region may have at least 50% sequence identity in its amino acid sequence to homologous sequence regions of a vertebral, preferably mammalian (as defined above), preferably a human, antibody.

According to a preferred embodiment, the constant region is of vertebral, preferably mammary origin, particularly preferred of human, canine, murine, equine, goat and/or camel origin. The constant region may have at least 50% sequence identity in its amino acid sequence to homologous sequence regions of a vertebral, preferably mammalian (as defined above), preferably a human, antibody.

The present invention in one preferred embodiment is embodied by providing a nucleic acid sequence with an increased expression rate for the production of recombinant proteins, provided with at least one expression cassette for the expression of one or more peptides. Herein, the expression cassette according to the invention comprises at least one promoter element and at least one first transcription unit coding for a protein, wherein the promoter element consists of the nucleic acid sequence of SEQ ID NO: 1 or a nucleic acid sequence with a homology of at least 70%, preferably at least 80%, particularly preferably at least 90%, very particularly preferably at least 95%, further preferably at least 99% to SEQ ID NO: 1.

The term “promoter” as used in the present invention refers to a polynucleotide sequence that is localized upstream of a gene and regulates the transcription of a functional gene. The promoter forms a recognition and binding site for an RNA polymerase which initiates transcription of the gene.

The term “homology” as used in the invention refers to the similarity between nucleotide sequences of DNA or RNA and/or between amino acid sequences of proteins.

In a preferably embodiment, the SEQ ID NO:1 represents a promoter, hereinafter referred to as HASP1mod, which has been found to be particularly suitable for regulated protein production. Herein, the HASP1mod promoter is a promoter element derived from the natural HASP1 promoter, wherein a partial sequence of the natural HASP1 promoter has been duplicated.

The SEQ ID NO:1 is as follows, wherein the underlined portion represents the duplication:

5′-CATACAGTGAATGTAACTTTCGAATTGACAGTATTAGTAGTCGT
ATTGACAGTGAGGCACGCCCCTCAATGTGCGAGGTGGAAAATATACC
AGCATGACAATGAATCTTGGAGATTCTTTTGCTGTCATCAAGATTCA
CCGCCAAATCTTCAGGAACCTATCACGTCCACAGGCGATGTTAATTC
TTGAGTCGTCAAAACAAAGTCCTGTCCTACCTGTAGAAGTTGACAGC
GAGCAATTGTATGCAAACTTCTGACTTTGTTATAATAACATTAAAGG
TAATTAAGTATCTTCAATTAGGCATTTTGTCACTGTCAGTCCGTTCC
GACAATATAGGTAGATTTGGAATGAATCTTTTCTATGCTCATACAGT
GAATGTAACTTTCGAATTGACAGTATTAGTAGTCGTATTGACAGTGA
GGCACGCCCCTCAATGTGCGAGGTGGAAAATATACCAGCATGACAAT
GAATCTTGGAGATTCTTTTGCTGTCATCAAGATTCACCGCCAAATCT
TCAGGAACCTATCACGTCCACAGGCGATGTTAATTCTTGAGTCGTCA
AAACAAAGTCCTGTCCTACCTGTAGAAGTTGACAGCGAGCAATTGTA
TGCAAACTTCTGACTTTGTTATAATAACATTAAAGGTAATTAAGTAT
CTTCAATTAGGCATTTTGTCACTGTCAGTCCGTTCCGACAATATAGG
TAGATTTGGAATGAATCTTTTCTATGCTGCTGCGAATCTTGTACACC
TTTGAGGCCGTAGATTCTGTCCGACGAAGCGATAATTATTGCAAAAT
ACATGGACTCATTATTTTGATTCGATTTCTTTTTGGTATCCGACTCG
AAAAGATCCATCACGGCGAGC-3′

In a preferably embodiment, the present invention comprises providing nucleic acid sequences whose promoter element has individual HASP1 sequence segments repetitively, for example between −100 and −1, between −200 to −101, between −300 to −201, between −400 to −301 and/or between −500 to −401 with respect to the start codon ATG. Here, these sections can be combined in any constellation and copy number. This results in a new sequence with less than 85% homology to the native HASP1 promoter.

According to a preferred embodiment of the present invention, at least one transcription unit comprises a polynucleotide encoding an amino acid sequence of SEQ ID NO: 2 or an amino acid sequence having a homology of at least 70%, preferably at least 80%, particularly preferably at least 90%, very particularly preferably at least 95%, further preferably at least 99% to SEQ ID NO:2.

The SEQ ID NO:2 represents an amino acid sequence of the hinge region of an antibody, which was derived from equine immunoglobulin sequences and has proven to be particularly protease-resistant. The use of this protease-resistant hinge region significantly reduces the proteolysis of antibodies produced in different formats and from different species, both in vivo and in vitro.

The SEQ ID NO:2 is as follows:
VIKEPCCCPKCP

In a preferably embodiment of the invention, a vector or an isolated nucleic acid comprising a nucleic acid according to the invention in simple or repetitive form can be provided, as well as the provision of a cell comprising a vector according to the invention or an isolated nucleic acid according to the invention or a nucleic acid sequence according to the invention or an amino acid sequence according to the invention.

Furthermore an amino acid comprising SEQ ID NO: 2 or an amino acid sequence having a homology of at least 70%, preferably at least 80%, particularly preferably at least 90%, very particularly preferably at least 95%, further preferably at least 99% to SEQ ID NO: 2 is provided.

According to the invention, the cell is a photosynthetically active cell, particularly a unicellular plant, preferably a diatom.

Preferably, the recombinant antibody in the hinge region is provided with a cysteine-rich amino acid sequence comprising at least 20 amino acids, preferably at least 15 amino acids, particularly preferred at least 12 amino acids, wherein at least 33% of the amino acids comprise cysteine. According to a particularly preferred embodiment, the amino acid sequence in the hinge region comprises or consists of (a) the sequence VIKEPCCCPKCP or (b) a sequence identity deviating from this amino acid sequence by a maximum of 30%, in particular by a maximum of 20%, particularly preferred by a maximum of 15%, or (c) an amino acid sequence which has only one amino acid substitution compared to the variant according to (a).

In a particularly preferred embodiment, the production rate amounts to from 20 mg/L to 1000 mg/L, especially preferably from 30 mg/L to 1000 mg/L of antibodies per liter of culture. This high production rate is absolutely necessary for technical utilization of the antibodies, as separation and purification at values below 20 mg/L is not technically practicable and is therefore absolutely necessary for economic applicability at a reasonable purification cost. These values are in the typical range for commercially used animal CHO (Chinese Hamster Ovary) cell cultures and thus far above the capacity achieved to date for diatoms, microalgae, unicellular plants and/or Viridiplantae. The realization that diatoms or other microalgae with their production capacities could become real competitors to CHO cells was previously unforeseeable.

Particularly preferred is an embodiment in which recombinant antibodies are obtained in a concentration of 20 mg/L to 1000 mg/L, more preferably 30 mg/L to 800 mg/L, alternatively at least 30 mg/L to 160 mg/L culture of a diatom or unicellular plant. This high production rate is the result of the inventors' discovery that various modifications to a diatom can lead to an unexpectedly high increase, for example by a factor of at least over 100 compared to the prior art. This allows the technical feasibility of obtaining and purifying antibodies from diatoms, which have a high purity and homogeneity. The production rate in the diatom could be increased from the conventional maximum of 3 mg of antibodies per liter of culture to, for example, at least 160 mg of antibodies per liter of culture. Particularly preferred, the production rate in the diatom can be increased from a conventional maximum of 3 mg antibody per liter of culture to 300 mg antibody per liter of culture, which corresponds to a factor of 100. In some very preferably embodiments, the production rate can be increased up to 1000 mg/L culture, which corresponds to a factor of 333.

A culture within the meaning of the present invention comprises the diatoms, culture medium and all other additives necessary for the provision of the recombinant antibodies according to the invention.

A culture medium according to the present invention comprises a liquid, preferably an aqueous, salt-containing liquid, which provides suitable nutrients, temperatures, pH values and other conditions that promote the growth and proliferation of cells, particularly preferably of microalgae, even more preferably of diatoms.

In some embodiments of the invention, the recombinant antibody is provided at a concentration of at least 100 mg/L of culture, more preferably at least 250 mg/L; most preferably at least 500 mg/L. Here, the previously reported values are exceeded by a factor of at least 33, more preferably by at least 83, most preferably by a factor of 166.

According to a preferred embodiment of the immunoassay, the glycosylation of the first antibody and/or the second antibody has a modified glycosylation pattern compared to the corresponding native antibody, preferably the glycosylation has a more homogeneous pattern with a homogeneity factor in the range from 1 to 3, particularly preferred the glycosylation has a homogeneous, mannose-rich N-glycan pattern with a homogeneity factor in the range preferably in the range from 1 to 3.

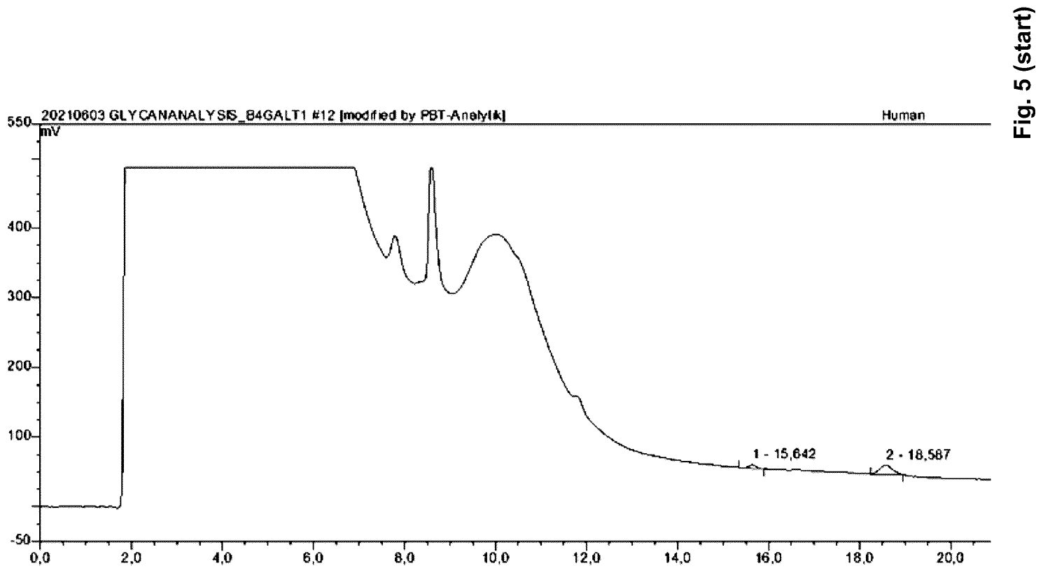

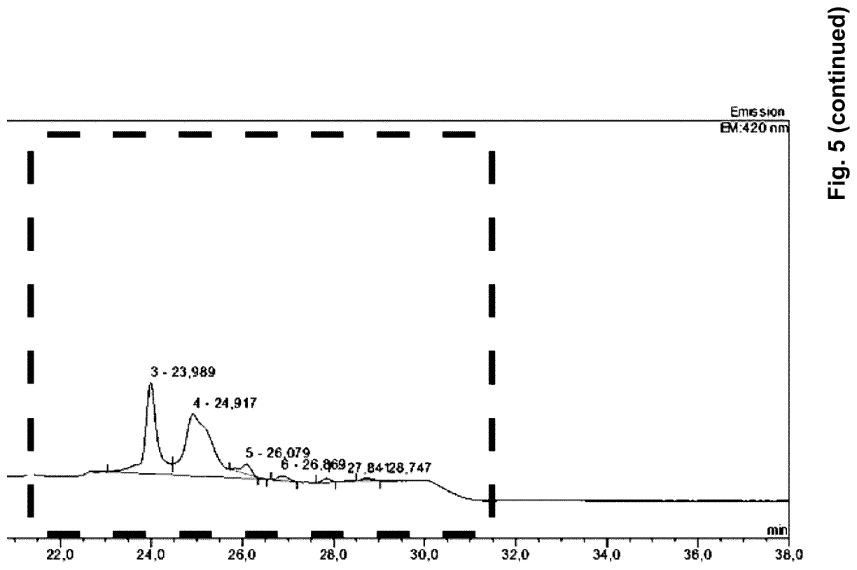

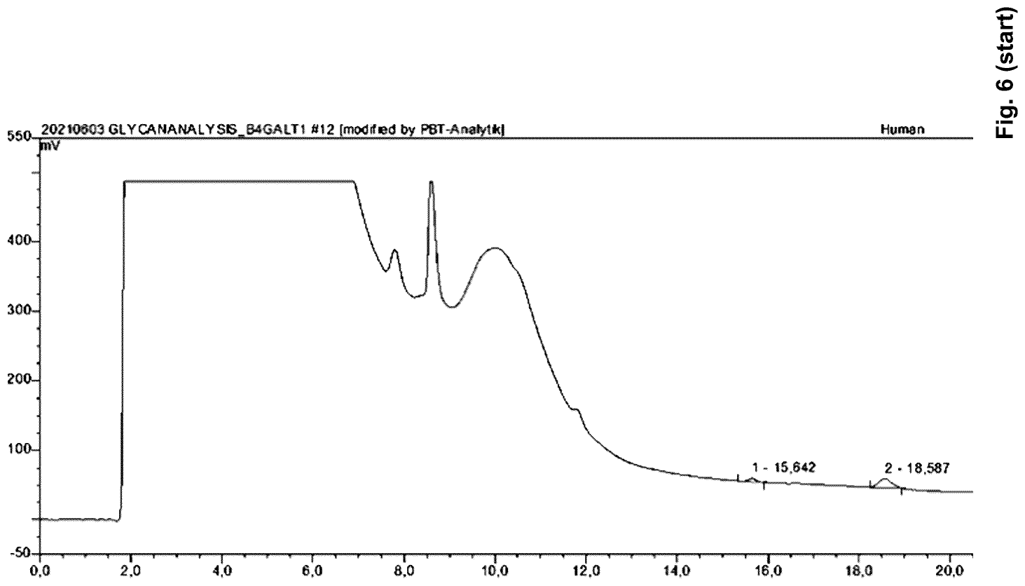

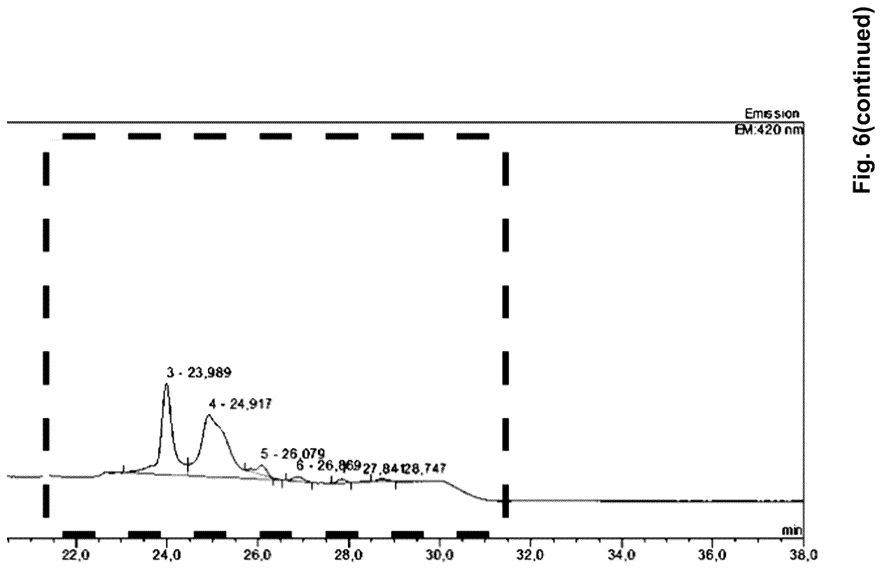

By the homogeneity factor in the sense of the present invention is meant the ratio of the number of baseline-separated, defined peaks in the chromatogram, determined by HPLC (High-Performance Liquid Chromatography) and/or UPLC (Ultra-Performance Liquid Chromatography) coupled with MS and/or HRMS and/or UV/Vis and/or diode array, between an antibody in the sense of the present compound, preferably an antibody expressed from a diatom, and the corresponding native antibody and/or an animal antibody. A homogeneity factor of 1 means that there is 1 peak less, of 2 means that there are 2 peaks less and so on. In a particularly preferred embodiment (FIGS. 5-7), a homogeneity factor of 3 is achieved; the comparative antibodies, which originate from a human cell culture (FIGS. 5 and 6), each have 6 peaks, whereas the recombinant antibody according to the present invention has only 3 peaks.

In a preferably embodiment of the immunoassay, the glycosylation of the first antibody and/or the second antibody exhibits an increased homogeneity of the glycosylation pattern compared to the corresponding native antibody, preferably a homogeneous, mannose-rich N-glycan pattern, without requiring additional addition of mannose to the culture medium.

The differences in glycosylation patterns of antibodies according to the present invention from mammalian systems result from the presence of different “mannose-rich” N-glycans (from mannose-5 to mannose-9). However, in contrast to natural mammalian cells, which have a relatively low mannose content, commercially used cell cultures such as CHO cells have a higher mannose content due to the deliberate addition of mannose. CHO-based antibodies often exhibit a markedly heterogeneous distribution of glycosylation. In FIG. 5-7 a clear differentiation can be seen. The consistency of glycosylation is often not given in CHO, Expi and other animal or human cells or cell cultures (recognizable by the numerous peaks in the framed area of FIG. 5).

This inconsistency affects the specificity and stability of the antibodies. Therefore, mannose is often added to the medium of animal or human cell cultures in order to achieve greater uniformity (despite the associated disadvantages). Diatoms are naturally high in mannose. Furthermore, our antibodies show remarkable homogeneity, i.e. antibodies derived from different cells in a culture show uniform glycosylation patterns (FIG. 5). Thus, the probability of undesirable properties such as the detection of foreign proteins and fluctuations in antibody stability is considerably reduced.

Recombinant antibodies are today either produced in animals or by means of human or animal-based expression systems, i.e. eukaryotic cell lines. Here, mammalian cell systems are preferred, especially when complex proteins are involved, such as antibodies, for which complex post-translational modifications are essential for their analytical or therapeutic efficacy. Among these post-translational modifications are the glycosylation patterns of antibodies. A major problem with currently produced recombinant proteins is that they have inconsistent glycosylation patterns. Thus, recombinant antibodies from currently used expression systems, particularly from individuals, are often hyper-glycosylated, i.e., for example, more mannose residues are inserted, which are often additionally provided with unusual branches. These can “break off” (degrade) and/or this can lead to the proteins not being effective, or that unwanted side reactions occur by the immune system.

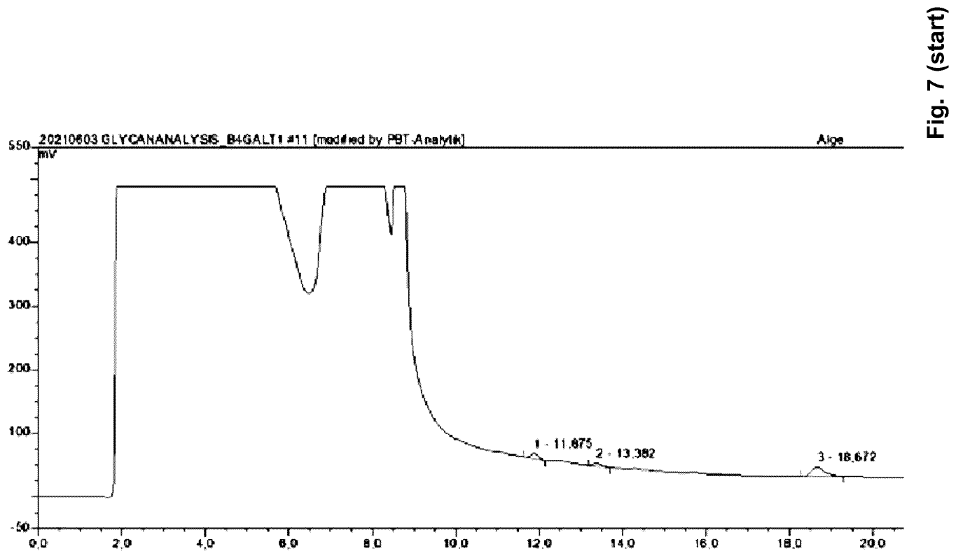

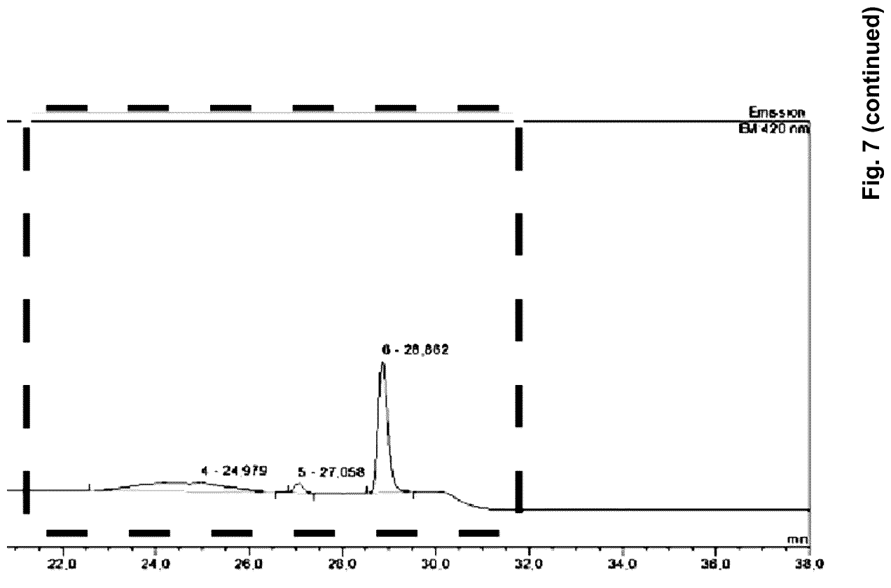

According to a preferably embodiment of the invention, the glycosylation (also referred to as glycosylation pattern) of the antibody (as defined herein), e.g. the first antibody and/or the second antibody and/or any further antibody, differs from the corresponding native antibody as present in the individual. Particularly preferred, the glycosylation pattern of the recombinant antibody (as defined herein) is more homogeneous than the glycosylation pattern of the native antibody as expressed in an individual (as defined herein) (cf. e.g. FIGS. 5 and 6 compared to FIG. 7). As such, the glycosylation pattern of the recombinant antibody (as defined herein) is provided with fewer branches compared to the native antibody, for example, which can “break off” (degrade) as mentioned above and/or (in combination) can lead to the antibodies not being effective or to undesirable side reactions by the immune system. A recombinant antibody disclosed herein (i.e. an antibody obtained from diatoms, unicellular plants or viridiplantae) represents a so-called biosimilar. Preferably, such antibodies whose glycosylation pattern differs from the corresponding native antibody are preferred as a marker for a disease of the individual or as a marker for the physiological state of the individual.

In a preferably embodiment of the immunoassay, the recombinant antibody is a mosaic antibody, wherein the mosaic antibody comprises at least a first sequence selected from at least a first organism, and at least a second sequence selected from at least a second organism. The first and second organisms are different organisms. The first sequence can be the heavy chain or part of the heavy chain, the second sequence can be the light chain or part of the light chain. The hinge region as a defined section of the heavy chain sequence can either originate from the same organism as the rest of the heavy chain sequence or preferably from a different organism.

In an alternatively embodiment, the first sequence may be selected from a first organism and a second organism. In a further alternatively embodiment, the second sequence may be selected from a first organism and a second organism.

The provision of mosaic antibodies leads to novel and non-naturally occurring antibodies. These antibodies are carefully designed using in silico processes, resulting in sequences that do not occur in nature. Process optimizations have led to the creation of antibody regions that have sequence matches to database sequences from a range of animal and human sources. Consequently, the antibodies produced are mosaic antibodies or mosaic proteins, which contain genetic sequences from different species.

The invention represents a deviation from the conventional methods for producing chimeric antibodies. Although chimeric antibodies have already been produced in animal cell cultures, this approach requires the use of transgenic animals. In contrast the invention described in the patent enables the production of chimeric antibodies in diatoms, so that no transgenic organisms, particularly no transgenic animals, are required.

The antibodies synthesized using this method, for example, have a basic structure that corresponds to the sequence of the human immunoglobulin IgG4.

In a preferably embodiment, this mosaic protein may have the following outlined structure consisting of a heavy chain, light chain and hinge region:

### A) Heavy Chain (Exemplary Structure):

C_{H}1-C_{H}3-without hinge region at least 80% homologous to human IgG4; for example, C_{H}1 without hinge comprises 116-118 amino acids, C_{H}2 and C_{H}3 together 215-220 amino acids, wherein the hinge region usually comprises 10-14 amino acids, the 80% homology refers to the minimum 331 and maximum 338 amino acids of C_{H}1, C_{H}2 und C_{H}3, which at 80% results in a number of 264-270 amino acids.
Hinge region corresponds to at least 80% of the protected sequence, further templates for the hinge region, which is particularly resistant to proteolytic degradation, and preferably originates from the order Perissodactyla, particularly preferably Equidae.
Variable chain is homologous to the IgG sequences of very different organisms, preferably selected from the list consisting of human, mouse and rabbit.

### B) Light Chain (Example Structure):

The constant region of the light chain is homologous to those of the order Perissodactyla, particularly preferably Equidae.
Variable chain is homologous to the light chain IgG sequences of very different organisms, preferably selected from the list consisting of human, mouse, rabbit, but particularly preferred to variable chains of human kappa-type light chains. Here, too, it was found that these have an influence on the production quantities and protease resistance.

Preferably, the different combinations lead depending on the sequence used to an increased or decreased production rate of the target protein or antibody.

An important feature of the invention is the variability of the domain combinations within the sequences. Depending on the selected sequence arrangements, different production rates for the target protein are achieved. This flexibility makes it possible to adjust the protein production to the desired level.

In summary, the present disclosure provides an approach for producing antibodies using diatoms that yields non-naturally occurring mosaic antibodies and/or mosaic proteins having sequences from multiple species. This innovation bypasses the dependence on transgenic animals and offers benefits such as customizable protein designs, improved production control and potential applications in various fields, including diagnostics and therapeutics. In particular, properties such as stability, selectivity and production rate can be specifically optimized and controlled through the targeted design of these antibodies.

In a preferred embodiment, the immunoassay according to the present invention comprises at least one further antibody and/or auxiliary protein, wherein preferably the further antibody and/or auxiliary protein is obtained from a diatom and/or unicellular plant and/or Viridiplantae and particularly preferably is vegan.

Vegan in the sense of the present invention, explicitly a vegan immunoassay in the sense of the present invention, is an assay which is produced by biotechnological methods and methods in compliance with the principles of veganism. This means that these products are developed without the use of animal materials and/or animal cell cultures or by-products and that no animal testing is carried out during their manufacture.

The immunoassay may provide, in addition to the first and/or second antibody, another antibody and/or another auxiliary protein. Auxiliary proteins serve, for example, as blockers to saturate the free surfaces (i.e. areas where antibodies are not immobilized and unspecific binding can occur) of a reaction vessel or membrane. For example, the auxiliary protein is bovine serum albumin (BSA), a casein, a modified BSA or a modified casein that acts as a blocker, e.g. to saturate free surfaces.

Auxiliary proteins (as defined herein) can perform multiple functions:
Saturation of the membranes: the membrane (e.g. the nitrocellulose membrane) binds proteins of any origin with high efficiency. Therefore, antibodies can be firmly bound to the membrane in an immunoassay, e.g. an LFA. Between the areas where antibodies saturate the membrane, however, there are still membrane regions where no protein is bound. Proteins from the sample to be tested would bind to them and thus lead to false results. If the antigen is immobilized (e.g. in ELISA), the antibodies used in the test would bind specifically to the antigen, but also non-specifically to the surface. An evaluation would not be possible. These proteins block the protein-binding surfaces so that antibodies can only bind specifically to their antigens and therefore play a central role in immunoassays.
These auxiliary proteins are also helpful during the incubation of antibodies and antigen. The excess of auxiliary protein, e.g. BSA and/or casein from the milk, ensures that proteases contained in the sample that could degrade antigen or antibodies are also offered BSA or casein as a substrate for proteolysis. As there is an excess of casein/BSA in the solution, the more BSA there is in the solution, the lower the risk (statistically) that an antibody will be proteolytically damaged by chance.

According to a particularly preferred embodiment, the immunoassay may also comprise another antibody and/or one or more further auxiliary proteins in addition to the recombinant antibody. In general, auxiliary proteins are used, for example, to saturate (block) free binding sites on the matrix (i.e. areas where no proteins from the sample to be analyzed are yet immobilized). Since the matrices generally bind proteins and antibodies are proteins, antibodies would bind non-specifically to such free areas and could no longer bind to their immobilized antigens. Therefore, in all immunoassays, such areas of a reaction vessel, microtiter plate or membrane are saturated with one or more auxiliary proteins. Often, particularly in the prior art, such auxiliary proteins of animal origin, such as bovine serum albumin (BSA), are caseins, which saturate free surfaces on the matrix as a blocking agent. Casein(s) and casein(s) are used interchangeably.

In a particularly preferred embodiment of the invention, auxiliary proteins (as defined herein) may perform multiple functions:
Membrane saturation: the membrane (e.g. the nitrocellulose membrane) binds proteins of any origin with high efficiency. Therefore, proteins to be analyzed in an immunoassay, e.g. antibodies in an LFA, can be firmly bound to the membrane. However, between the areas where proteins have bound to the membrane, there are still membrane regions where no protein is bound. In an immunoassay such as ELISA or immunoblot, the antibodies would be bound to them and could no longer detect their antigens. With LFAs, proteins from the sample to be tested would be bound by these free membrane regions and thus lead to false results. These auxiliary proteins block the protein-binding surfaces so that antibodies can only bind specifically to their antigens and therefore play a central role in immunoassays.
These auxiliary proteins are also helpful during the incubation of antibodies and antigen. The excess of auxiliary protein, e.g. BSA and/or casein from the milk, ensures that proteases contained in the sample, which could degrade antigen or antibodies, are also offered BSA or casein as a substrate for proteolysis. As there is an excess of casein/BSA in the solution, the more BSA there is in the solution, the lower the risk (statistically) that an antibody will be proteolytically damaged by chance.

Conventionally obtained BSA and casein are of animal origin. Although it is initially available at low cost, the purification of BSA requires a great deal of technical effort. In particular, the removal of (human) pathogenic viruses and prions (e.g. BSE, “mad cow disease”) requires great effort. Other sources, such as bacteria or yeasts, cannot produce BSA heterologously because it is not only glycosylated but also contains several post-translationally modified amino acids. The extraction of vegan BSA from diatoms, unicellular plants or viridiplantae, particularly from diatoms, as disclosed herein therefore has the advantage that purification and removal of (human) pathogenic viruses and prions are not required.

The further antibodies or excipients are preferably recombinant antibodies or recombinant proteins, respectively, which are obtained (also as defined herein) from a diatom, unicellular plant or Viridiplantae and therefore comprise a heterologous diatom-, plant- or microalga-specific signal peptide and/or a glycosylation pattern different from that of a native antibody obtained from an individual (as defined herein).

In a particularly preferred embodiment, further antibodies or excipients are recombinant antibodies or recombinant proteins, respectively, which are obtained (also as defined herein) from a diatom or unicellular plant and therefore have a heterologous diatom-, plant- or microalga-specific signal peptide and/or a glycosylation pattern deviating from that of a native antibody obtained from an individual (as defined herein).

In a preferred embodiment of the immunoassay, the amino acid sequence of the first antibody and/or the second antibody has a vertebral, preferably a mammalian, particularly preferred a human antibody; or it has an amino acid sequence or consists of an amino acid sequence which has at least 80%, preferably at least 85%, particularly preferred at least 90%, most preferably at least 95%, particularly at least 97% sequence identity with homologous sequence regions of a vertebral and/or mammalian and/or human antibody. In this way, compatibility with a range of targeted antigens can be ensured and a high quality of antibodies can be achieved.

In a preferred embodiment of the present invention, the nucleic acid sequence encoding for the first antibody and/or the second antibody is codon optimized for the host organism from which the first antibody and/or the second antibody is derived, preferably codon optimized for Phaeodactylum tricornutum.

When performing codon optimization, the base sequence is also adjusted at the same time, for example to facilitate the cloning of the recombinant DNA. One aspect of this is the removal of unneeded or undesired recognition sites of restriction enzymes in the recombinant DNA.

The invention therefore also comprises a nucleic acid encoding a first antibody, a second antibody, a further antibody and/or an auxiliary protein, wherein the sequence of the nucleic acid is codon optimized for expression in a diatom, unicellular plant or Viridiplantae, particularly in a diatom.

The invention therefore preferably also comprises a nucleic acid which codes for a first antibody, a second antibody, a further antibody and/or an auxiliary protein, wherein the sequence of the nucleic acid is codon optimized for expression in a diatom or unicellular plant, in particular in a diatom.

The inventors have also developed a method that does not take into account—as is usually the case—each codon individually for the codon optimization of the sequence. Rather, a position-specific matrix is used to create a profile of the codon frequency over the entire original sequence and this profile is used to transfer this codon frequency at each position to the sequence codon optimized for diatoms. In this way, the folding of the antibody chains is particularly optimized directly after translation, as areas of the antibody chains that are difficult to fold are translated somewhat more slowly than areas that are easy to fold. This has a direct effect on the quantity of antibodies produced and is also relevant for the high homogeneity of the antibodies produced in diatoms.

Moreover, a codon optimized sequence of a nucleic acid for expression in a host organism (as defined herein) has the advantage that the folding of the antibody is improved to correspond to the native counterpart of the antibody, resulting in increased stability of the antibody and increased biological activity of the antibody expressed in the host organism (as defined herein).

Furthermore, a codon optimized sequence of a nucleic acid has the advantage that the expression rate in the host organism compared to a non-codon optimized sequence of a nucleic acid is increased at least by a factor of 10, preferably at least by a factor of 20, particularly preferred at least by a factor of 30, most preferably at least by a factor of 40. For example the production rate in the diatom can be increased from a conventional maximum of 3 mg of antibodies per liter of culture to 160 mg of antibodies per liter of culture. Particularly preferred, the production rate in the diatom can be increased from conventionally a maximum of 3 mg antibody per liter of culture to 100-1000 mg antibody per liter of culture. Particularly preferably, the production rate in the diatom can be increased from a conventional maximum of 3 mg antibody per liter of culture to 300 mg antibody per liter of culture, which corresponds to a factor of 100. In some very preferably embodiments, the production rate can be increased up to 1000 mg/L culture, which corresponds to a factor of 333.

Preferably, the recombinant antibody according to the invention is modified in the hinge region in such a way that it has increased stability, preferably with a stability factor of 1.1 to 5, against diatom-, plant- or microalga-specific proteases compared to the native antibody. This increases the yield of antibodies during purification (also referred to as the downstream process) and produces fewer interfering degradation products that reduce the purity of the antibodies obtained from cultivation, resulting in a more homogeneous recombinant antibody as defined in the present invention from cultivation that provides more specific signals and fewer cross-reactions.

The increased stability is related to the stability of diatoms' own enzymes, especially proteases, which can attack the region of the antibody known as the hinge region and cleave it enzymatically. A stability factor is associated with the increased time that the antibody with the modified hinge region is stable to proteases under cultivation conditions, which increases the production quantities, particularly the yield of functional recombinant antibodies after separation. Since there are fewer proteolytically cleaved antibody fragments, the antibody solution is more homogeneous and allows more specific detection with significantly fewer cross-reactions.

In a preferably embodiment, the stability factor between a native hinge antibody and a recombinant antibody with modified hinge according to the present inventions is preferably determined in an SDS PAGE. The stability factor can be determined via degradation products of the recombinant antibody, which lead to additional bands in the Coomassie staining. By adding certain proteases, preferably diatom-, animal-, human-, plant- or microalga-specific proteases, to the recombinant antibody with modified hinge and the antibody with native hinge and performing SDS-PAGE at certain time intervals, it is possible to determine how much intact antibody is present after this time and how many proteolytically generated fragments are detected in the SDS-PAGE. The absolute proportions can be determined densitometrically and the quotient of the concentration of the recombinant antigen and the native antibody gives the stability factor. In a particularly preferred embodiment, the stability factor is between 1.1 and 10, particularly preferably between 1.1 and 5, most preferably between 1.1 and 3.

According to a preferably embodiment of the invention, the amino acid sequence of the antibody, for example of the first antibody and/or of the second antibody and/or of each further antibody, is modified in such a way that it has an increased stability towards diatom-, animal-, human-, plant- or microalga-specific proteases.

For example, the amino acid sequence of the antibody, e.g. of the first antibody and/or the second antibody and/or each further antibody, is modified in the hinge region in such a way that it has increased stability with respect to host-specific, particularly diatom-, plant- or microalga-specific proteases. In this way, the stability of the antibody in the host organism in which the antibody is expressed and thus also the yield of intact antibody from the culture can be increased.

Particularly preferred is an embodiment of the immunoassay, wherein the recombinant antibody is expressed from a stably transformed diatom or unicellular plant, preferably from a stably transformed diatom, for several generations, preferably for at least 60 generations, more preferably for at least 80 generations, most preferably for at least 100 generations. Thus, high yields can be guaranteed with a simultaneously high quality of the antibody, whereby the quality of assays over different batches can be ensured.

A generation in the sense of the present invention concludes with a cell division and denotes a unit of cellular replication. For instance, 60 generations correspond to 60 consecutive cell divisions starting from one initial cell. Analogously, “generation time” refers to the time interval between two successive generations of organisms in a population. It is the time it takes for a single cell or organism to divide and give rise to two new cells or organisms. The generation time is a basic parameter that characterizes the growth rate of unicellular organisms. It provides information on how quickly a population of genetically identical cells can multiply under optimal conditions (mitosis). Shorter generation times indicate faster growth rates and higher reproductive capacities, while longer generation times indicate slower growth rates and potentially more complex cellular processes.

In a preferably embodiment of the invention, the generation time is between 6 and 48 hours, preferably between 12 and 24 hours. This generation time is significantly shorter than that of higher Viridiplantae known from the state of the art, such as N. tabacum. Due to the short generation time, a faster growth of the culture and thus a significantly higher production capacity for antibodies can be achieved than is possible with higher plants.

Transiently modified higher plants are genetically modified plants in which foreign genetic material, such as genes coding for certain proteins or traits, is introduced into the plant cells for a short period of time. This change is temporary and does not lead to the integration of the foreign genes into the genome of the plant. instead, the foreign genes are expressed and the desired properties are only produced for a limited time. This information is thus lost after one generation, which leads to a high effort in the cultivation of cell cultures as well as in the control of product quality. Instead, the antibodies according to the present invention can be stably expressed over numerous generations, which enables high quality and controllable conditions.

A stably transformed culture according to the invention enables the expression of the antibody over at least 60 generations, preferably over at least 80 and particularly preferred over at least 100 generations.

In a preferred embodiment, the antibody is stably expressed in a culture for at least 30 days, more preferably for at least 40 days and most preferably for at least 60 days after inoculation of the culture.

In addition to the other advantages over higher plants that produce antibodies transiently or stably, diatoms do not contain fibers, which greatly facilitates the purification of antibodies.

Particularly preferably, an antibody in the context of the immunoassay according to the present invention, is expressed intracellularly, preferably in a stably transformed diatom. So far, in the state of the art, extracellular secretion has been used to obtain antibodies from diatoms, as the antibodies are released directly into the culture medium. Theoretically, this would lead to easier isolation, but the authors made the unexpected finding that intracellular secretion leads to significantly higher production levels.

This outstanding achievement of the inventors, together with the other properties of the antibodies described here, led to a dramatic increase in antibody production, preferably by a factor of 100 or more.

In a preferably embodiment, the first and/or the second antibody may be an antibody directed against human chorionic gonadotropin (hCG), preferably the antibody is an hCG antibody.

hCG is a glycoprotein hormone that is produced before the embryo implants and indicates pregnancy very early on, and is mainly produced by the placenta during pregnancy. It plays a crucial role in maintaining the corpus luteum, which in turn produces progesterone to support the early stages of pregnancy. Antibodies directed against hCG can be used in various applications including diagnostic tests for pregnancy. Pregnancy tests detect the presence of hCG in urine or blood, which provides with a pregnancy. These antibodies are used as recognition elements to bind to hCG molecules and produce a measurable signal that confirms pregnancy. This enables early detection of pregnancy, particularly as PoCT. PoC in the sense of the present invention means point-of-care, analogously PoCT means point-of-care testing, i.e. patient-oriented self-diagnostics. This concept refers to medical diagnostic tests that are performed close to the patient, usually outside the traditional laboratory environment.

In a preferred embodiment, the immunoassay with a recombinant antibody directed against the hCG protein is provided as a lateral flow immunoassay, particularly preferably as a kit. Thereby, the immunoassay can be provided close to the patient, which allows for simple, rapid on-site diagnostics.

In a preferably embodiment, the biologically active antigen is a part of the epitope of a virus, preferably a pathogenic virus, for example influenza, SARS-COV-2, RSV, adenovirus, Strep A, norovirus, rotavirus, HIV. Preferably, the immunoassay uses highly specific antibodies that recognize and bind to the selected part of the epitope, enabling rapid and accurate identification of viral infections. This approach facilitates early diagnosis, which enables early and targeted treatment, as well as continued close monitoring of infection progression and the ability to detect outbreaks quickly, ultimately contributing to timely public health responses and effective containment strategies.

In a preferably embodiment, the biologically active antigen is a tumor-associated sequence, preferably an HLA complex and/or a sequenced part of a tumor epitope and/or a tumor marker, for example IFN-γ, IL-8, PSA, CEA, AFP, DCP, CA 125, HER2/neu. This enables early detection and treatment of malignant degenerations and can therefore drastically increase a patient's chances of recovery. In a preferably embodiment as a lateral flow assay, it can serve as a PoCT for early diagnosis. In a particularly preferred embodiment as LFA or ELISA, the immunoassay serves as a diagnostic tool performed in laboratories or medical facilities by medical personnel.

IFN-γ (interferon-gamma) is a cytokine produced by immune cells in response to infections and plays a key role in immune responses against pathogens. An antibody-based immunoassay enables the precise detection of IFN-γ in patient samples and thus helps in the diagnosis of immune system disorders, the monitoring of autoimmune diseases and the assessment of response to immunotherapy.

IL-8 (interleukin-8) is a chemokine involved in inflammatory responses and the recruitment of immune cells. The detection of IL-8 using antibody-based immunoassays helps to understand inflammation-related conditions such as autoimmune diseases, allergies and infections and enables accurate monitoring and evaluation of treatment.

PSA (prostate-specific antigen) is a protein produced by the prostate. Elevated levels can indicate prostate problems including cancer. Antibody-based immunoassays provide an accurate measurement of PSA and help in the early detection of prostate cancer, risk assessment and monitoring the effectiveness of treatments.

CEA (carcinoembryonic antigen) is a glycoprotein that is found in elevated concentrations in certain types of cancer, particularly colorectal cancer. Antibody-based immunoassays enable the sensitive detection of CEA, which is helpful in diagnosing cancer, monitoring treatment progress and detecting possible relapses.

AFP (alpha-fetoprotein) is a protein that is formed during fetal development. Elevated levels in adults can indicate liver disease or certain types of cancer such as liver cancer. An antibody-based immunoassay enables the precise detection of AFP and thus supports early diagnosis and monitoring of treatment success.

DCP (des-gamma-carboxy-prothrombin) is a protein produced by liver cells. Elevated DCP levels are associated with liver cancer. Antibody-based immunoassays enable accurate measurement of DCP and thus help in the early detection of hepatocellular carcinoma and the monitoring of treatment success.

CA 125 (Cancer Antigen 125) is a protein that is elevated in some cancers, particularly ovarian cancer. Antibody-based immunoassays provide a reliable method for measuring CA 125 levels, which is helpful in diagnosing ovarian cancer, monitoring disease progression and assessing treatment success.

HER2/neu (human epidermal growth factor receptor 2) is a protein involved in the regulation of cell growth. Elevated levels are associated with certain aggressive breast cancers. Detecting HER2/neu using antibody-based immunoassays helps to identify appropriate treatment strategies, predict disease progression and monitor treatment success in breast cancer patients.

In a preferred embodiment of the immunoassay, the biologically active antigen is a characteristic sequence for identifying a protein, preferably an enzyme tag, particularly preferred selected from the list consisting of 6×His tag, Strep tag, c-Myk tag, Flag tag and

GST tag. This allows an immunoassay to detect these protein tags, providing the specificity and sensitivity to accurately quantify, purify and characterize proteins in a variety of research contexts.

The 6×His tag is a short peptide sequence with six histidine residues that is often genetically fused to proteins. This fusion tag enables efficient purification of the tagged protein using immobilized metal affinity chromatography (IMAC) due to the strong binding affinity between histidine and divalent metal ions, such as nickel. In an antibody-based immunoassay, specific antibodies can recognize and bind to the 6×His tag, facilitating the detection and quantification of the protein. The technical advantage of the immunoassay lies in its high specificity and sensitivity, which enables precise measurement of the labeled protein even in complex biological samples.

The Strep tag is a peptide tag characterized by an eight-amino acid sequence (WSHPQFEK) that has a high binding affinity for the streptavidin protein. When the Strep tag is fused to a target protein, it enables straightforward purification by interacting with streptavidin-coated surfaces. In an antibody-based immunoassay, antibodies that recognize the Strep tag can selectively bind to the tag of the protein of interest. This approach enables efficient and specific detection of the target protein, making it valuable for various research and diagnostic applications.

The c-Myc tag is derived from the c-Myc protein and consists of ten amino acids (EQKLISEEDL). It is commonly used as a fusion tag to facilitate the detection and purification of proteins. In antibody-based immunoassays, antibodies against the c-Myc tag can bind specifically to the tag, allowing sensitive detection and quantification of the labeled protein. The technical advantage of this technique lies in its versatility, as it can be used for a wide range of protein studies and tests.

The flag tag is a peptide sequence (DYKDDDDK) that is usually attached to the N- or C-terminus of a protein to facilitate identification and isolation of the protein. It is recognized by commercially available anti-flag antibodies and enables uncomplicated protein detection and purification. The technical advantage of an antibody-based flag tag immunoassay is its robustness and wide availability, making it a popular choice for researchers working with recombinant proteins.

The GST tag, derived from the enzyme glutathione S-transferase, is commonly used for protein expression, purification and interaction studies. The combination of the tag with glutathione-conjugated matrices enables efficient purification in one step. In antibody-based immunoassays, antibodies specific for the GST tag can detect and quantify the tagged protein. The technical advantage of this immunoassay lies in its simplicity and the possibility of achieving a high protein yield and purity through the affinity purification step.

In a preferably embodiment of the immunoassay, the biologically active antigen is a sequence associated with nutritional parameters, for example transcobalamin II, ferritin, homocysteine, eicosapentaenoic acid (EPA) and docosahexaenoic acid (DHA), or calcitriol. This makes it possible to check some important parameters which allow an individual or subject, preferably a human being, to check and improve his health. In particular, individuals with certain dietary habits may be deficient in certain vitamins and trace elements, wherein an immunoassay according to the present invention, preferably embodied as an LFA, particularly preferably as a PoCT, can help to detect and compensate for these deficiencies. In an alternatively embodiment, the result of the immunoassay is only a recommendation and not a medical indication.

Transcobalamin II (TCII) is a transport protein that plays a crucial role in the transportation of vitamin B12 (cobalamin) in the body. It binds to vitamin B12 and facilitates its transport to the cells for various biochemical processes. The presence of TCII can indicate a vitamin B12 deficiency or certain diseases. An immunoassay based on antibodies targeting TCII enables the accurate detection and quantification of this protein in clinical samples, helping to diagnose and monitor disorders related to vitamin B12 metabolism.

Ferritin is a protein that stores and releases iron in a controlled manner, contributing to iron homeostasis in the body. The measurement of ferritin levels is crucial for the assessment of iron status and the diagnosis of diseases such as iron deficiency anemia or iron overload. An immunoassay based on antibodies, preferably in the form of an LFA as a PoCT targeting ferritin, allows precise quantification of this protein in blood or tissue samples and provides valuable information about a person's iron levels and overall health.

Homocysteine is an amino acid that results from the metabolism of methionine. Elevated homocysteine levels in the blood are associated with an increased risk of cardiovascular disease and other health problems. The detection of homocysteine with an immunoassay, preferably in the form of an LFA as a PoCT, provides a reliable method for assessing an individual's cardiovascular risk and monitoring the effectiveness of interventions to lower homocysteine levels.

Eicosapentaenoic acid (EPA) and docosahexaenoic acid (DHA) are omega-3 fatty acids found in certain fish oils and are known for their potential health benefits, including cardiovascular and cognitive support. The measurement of EPA and DHA levels in the blood provides information about a person's omega-3 fatty acid status and helps to make dietary recommendations. An antibody-based immunoassay, preferably in the form of an LFA as a PoCT targeting EPA and DHA, allows accurate quantification of these fatty acids and helps in personalized dietary assessment.

Calcitriol is the active form of vitamin D and plays a crucial role in maintaining the calcium and phosphorus balance, bone health and various physiological processes. The monitoring of calcitriol levels is important for the assessment of vitamin D status and the diagnosis of diseases such as rickets and osteoporosis. A antibody-based immunoassay, preferably in the form of an LFA as a PoCT specific for calcitriol, enables the precise measurement of this hormone in blood samples, helping to assess a person's vitamin D status and determine appropriate interventions.

In a preferably embodiment, the recombinant antibody is obtained from a diatom. In a particularly preferred embodiment, the diatom is Phaeodactylum tricornutum. This enables the provision of an antibody having the properties disclosed herein, particularly high homogeneity and purity, which enable the provision of an immunoassay according to the invention.

Preferably, the immunoassay is a lateral flow immunoassay, which provides fluid-connected at least one sample application area, a conjugate area and a capture area, which are arranged on a membrane.

In a lateral flow immunoassay (herein also referred to as “LFA”), three antibodies are typically used:
an immobilized first antibody, which is located in the capture area (also referred to as the test zone) and is also referred to as the capture antibody. This antibody, like the second antibody (also known as the detection antibody), is also directed against the antigen, preferably against a different epitope of the antigen than the detection antibody;

a second antibody, which is preferably provided in the conjugate area and is also referred to as a detection antibody, which is directed against an epitope of the antigen, e.g. a hormone, a protein or a peptide, such as human chorionic gonadotropin, wherein the second antibody is preferably conjugated to marker particles, e.g. gold, silver, latex, carbon, nanoparticles or enzymes.

A further antibody which is preferably located in a control area (control zone), preferably immobilized, and is directed against a detection/control antibody.

Preferably, the first antibody (e.g. the capture antibody), the second antibody (e.g. the detection antibody) and/or the further antibody (e.g. the capture antibody in the control zone), particularly preferably all antibodies, are obtained from a diatom, unicellular plant or Viridiplantae and are characterized by the characteristics defined herein.

Particularly preferably, the first antibody (e.g. the capture antibody), the second antibody (e.g. the detection antibody) and/or the further antibody (e.g. the capture antibody in the control zone), particularly preferably all antibodies, are obtained from a diatom or unicellular plant and are characterized by the characteristics defined herein.

In a preferably embodiment, the LFA is provided as a PoCT. In a particularly preferred embodiment, the LFA is provided as a kit, comprising at least the LFA and instructions. This allows easy use by the patient himself and can thus allow faster diagnostics outside the healthcare system infrastructure.

Preferably the first antibody in one embodiment relating to LFA and sandwich ELISA is directed against a first domain of the biologically active antigen (as defined herein). Herein, preferably, in one embodiment relating to LFA and sandwich ELISA, the second antibody is directed against a second domain of the biologically active antigen (as defined herein).

Preferably, in one embodiment, referring to ELISA, the first antibody is directed against a domain of the biologically active antigen (as defined herein) and the second antibody is preferably directed against a domain of the first antibody.

In a particularly preferred embodiment, the present invention is carried out as an enzyme-linked immunosorbent assay (ELISA) immunoassay, wherein at least
a. a sample application area is provided for the application of a biological sample, which
b. is confined by a capture area which is designed as a vessel boundary and/or part of a vessel boundary, preferably as a microtiter plate, and
c. a conjugate area, which is spatially arranged in the sample application area.

ELISA (enzyme-linked immunosorbent assay) is a widely used laboratory technique for detecting and quantifying the presence of specific proteins or antibodies in a sample. In some forms of ELISA, a target antigen or antibody is immobilized on a solid surface, and then specific antibodies bound to enzymes are employed for detecting and quantifying the amount of antigen that correlates with the amount of antibody bound. In a sandwich ELISA, two different antibodies are used. The first antibody is immobilized on the ELISA plate and binds specifically to the antigen in the sample. The second (detection antibody) is labeled, e.g. with an enzyme, and binds to a different epitope on the same antigen. Among the technical advantages of the ELISA is its high sensitivity, which enables the detection of low concentrations of target molecules. It is versatile and suitable for a wide range of sample types and molecules, including proteins, peptides and small molecules. An ELISA can provide quantitative information about the quantity of the target molecule present in a sample. It is one of the most important tools in molecular biology and has become indispensable in analysis and diagnostics. In addition to the antibodies and the sample to be analyzed, a microtiter plate as a carrier material and a reading device, a so-called ELISA reader, are required. There are different ways of performing the test. In the simplest form, the antigen to be analyzed is pipetted into the wells of the microtiter plates, where the antigen binds firmly to the polystyrene of the microtiter plates. Remaining free binding sites on the polystyrene are saturated with a blocking reagent (e.g. vegan BSA) so that the subsequently added antibodies can only bind to their antigens and not to free binding sites on the polystyrene.

Sodium dodecyl sulphate polyacrylamide gel electrophoresis (SDS-PAGE) is a widely used laboratory technique for separating proteins according to their molecular weight. In SDS-PAGE, proteins are denatured and coated with the anionic detergent sodium dodecyl sulphate (SDS) to give them a uniform negative charge. They are then loaded into a porous polyacrylamide gel and exposed to an electric field, causing the proteins to migrate according to their size. Among the technical advantages of SDS-PAGE is its high resolving power, which enables the precise separation of proteins in complex mixtures. It provides quantitative and qualitative information on protein samples, helping to identify and characterize proteins. SDS-PAGE is compatible with various sample types and can process both denatured and, with some methodological modifications, native proteins.

Proteins separated in SD-PAGE in this way can be transferred to a protein-binding membrane, for example a nitrocellulose, using Western blot transfer. In this way, the previously separated proteins become accessible for the binding of antibodies, which are added after saturation of the membrane areas still free after the Western blot. As is usual in an immunoassay, a cascade of at least two different antibodies is often used. Due to the previously performed cascade, a signal amplification occurs as the second antibody binds to the first antibody. The second antibody is often coupled with an enzyme such as alkaline phosphatase (AP) or horseradish peroxidase (HRP) to make the binding visible. Furthermore, signal amplification often occurs as the second antibody can bind multiple times to the first antibody. After addition, depending on the amount of bound antigen or antibody, an insoluble product is formed by the enzyme reaction, which precipitates directly at the site to which the antigen has run in the SDS-PAGE and thus clearly identifies the antigen.

Alternatively preferably, the immunoassay is an enzyme-linked immunosorbent assay (ELISA), e.g. a direct ELISA, an indirect ELISA, a direct sandwich ELISA or an indirect sandwich ELISA, preferably the ELISA is a direct sandwich ELISA or an indirect sandwich ELISA.

According to a preferred embodiment of the present invention, the conjugate area, the sample application area and the capture area may be spatially located in the same environment, for example in a vessel, preferably designed as a well of a microtiter plate or test tube, and may be separated from each other by temporally separated additions and washing steps. In one embodiment associated with an ELISA according to the present invention, an antigen corresponding to a biological sample of an individual is immobilized on the wall of the vessel to form the sample application region. Subsequently, a first antibody directed against the biologically active antigen is added, which binds to the antigen and is thus immobilized on the wall of the vessel, forming the capture area. After an optional washing step, a second antibody is added, which is directed against the first antibody, forming the conjugate area. The areas are fluid-connected but separated in time. In one embodiment associated with a sandwich ELISA according to the present invention, the first antibody is first immobilized to the wall of the vessel to form the capture area. Subsequently, a biological sample of an individual is added, forming the sample application area. After an optional washing step, a second antibody directed against the biologically active antigen is added, forming the conjugate area. The areas are fluid-connected but separated in time.

In a particularly preferred embodiment, the second antibody is provided as an antibody-enzyme conjugate. This allows the detection of a first antibody and quantification of the signal by the enzyme in an ELISA when a soluble product is formed or, in an immunoblot, when the product of the enzyme reaction precipitates at the site of its formation.

In a preferred embodiment of the present invention, a first antibody, preferably obtained from a diatom, is directed against an antigen, and a second antibody, also preferably obtained from a diatom and coupled to an enzyme, preferably alkaline phosphatase (AP) or horseradish peroxidase (HRP), is either also directed against the first antigen (LFA or sandwich ELISA) or against the first antibody (ELISA or immunoblot). Preferably, the second antibody can bind to the first antibody multiple times, which can lead to signal amplification. Particularly preferred, the second antibody can bind to the first antibody between 1 and 10 times, most preferably between 1 and 5 times. The resulting signal amplification thus preferably has a factor of 1-10, particularly preferred 1-5. Due to this strong amplification, the ELISA can determine a low concentration of antigen or a small amount of first antibody and/or second antibody is necessary to allow detection above the detection limit. Due to the low concentration, corresponding antigens can, for example, be detected earlier in the course of the disease and thus contribute to rapid detection and treatment, which can, for example, interrupt chains of infection.

In an embodiment of the invention associated with an LFA, the invention preferably provides a device provided with: a container, in particular a housing, and an immunoassay according to the invention, in particular a lateral flow immunoassay, arranged therein.

The invention also relates to a device comprising a housing (8), for example a container, in particular a housing, and an immunoassay (as defined herein) arranged therein, in particular a lateral flow immunoassay.

According to a preferably embodiment, the housing of the device is formed essentially, particularly exclusively, from a cellulose material, preferably paper or paperboard. This makes it possible to dispense with the use of plastics.

According to a particularly preferred embodiment, the housing of the device is substantially, particularly e.g. more than 90%, made of bioplastics, preferably polylactic acid (PLA), polyhydroxyalkanoates (PHA) or starch-based plastics. As bioplastics in this context, we refer to plastics that are biodegradable, i.e. that can break down naturally into environmentally friendly substances, e.g. in composting plants or in nature.

Preferably, the housing of the device is formed from at least two layers of cellulose material, which at least partially enclose the membrane. Here, the ends of the layers of cellulose material can be placed on top of each other in such a way that they are flush with each other.

In a preferred embodiment, the membrane is surrounded by a housing within the meaning of the present invention consisting predominantly, preferably 90 to 100%, of cellulose fibers, which on the one hand is water-resistant, preferably at least for transport and service life, and on the other hand is made from sustainable, preferably recovered plant fibers and is biodegradable, for example by microbiological degradation in the environment or by composting.

Biodegradable in the sense of the present invention means that at least 90-100% of the components, preferably 95-100% of the components, most preferably 99-100% of the components are biodegradable, for example by microbiological degradation in the environment and/or preferably by industrial composting according to EN 13432.

In a preferably embodiment, markings (symbols, characters, geometric shapes) are arranged on the housing at the membrane to allow easy interpretation of the immunoassay result. Particularly in the embodiment of a kit in which instructions are provided in addition to the assay according to the invention, the ease of use is also given for non-medical personnel. This means that the invention can be used as a PoCT.

For example, the ends of at least two layers of the cellulose material, which preferably form the housing of the device, are embossed and/or punched at the side areas where they contact each other, whereby they are firmly connected to each other. For example, the ends of the layers are joined together by an embossed seam (9).

According to a preferably embodiment of the invention, the sample application area is only partially arranged in the housing. The sample application area can be designed as a pad (cushion). The pad can consist of a bulky, porous or fibrous material that is adapted to absorb liquid quickly.

According to a preferably embodiment of the invention, the membrane has a fixing area (7) in the distal area (viewed in the direction of flow from the application area at the proximal end of the membrane), preferably distal to the capture area, by means of which the membrane is fixed in the housing of the device.

In the distal region of the membrane, a break point (10) can be provided on the housing of the device as an example of a separation region, through which the distal end of the housing can be separated, preferably with the distal end of the membrane.

Particularly preferred the device is provided such that the container, preferably a housing, is designed from sustainable and water-resistant materials, preferably paper and/or fiber cast. This allows the provision of an environmentally friendly immunoassay, particularly an environmentally friendly LFA immunoassay. Pregnancy tests alone produce approximately 900 tons of plastic each year, so providing a housing that does not use plastic but is designed from paper or fiber cast is desirable.

Preferably, the invention provides a kit provided with a device and instructions for performing the immunoassay. Particularly preferred, the kit contains further components, e.g. cotton swabs or other sampling utensils, buffer solution as a running medium mixture, further solutions, dessicates for drying and other aids known to the skilled person for carrying out an immunoassay as a PoCT or test kit for medically trained personnel.

Further, the present invention also relates to a kit comprising a device defined herein and instructions for performing the immunoassay arranged in the device.

Furthermore, the present invention also relates to the use of an immunoassay (as defined herein) for detecting a biologically active protein in a biological sample from an individual.

Also encompassed by the present invention is a method for detecting a biologically active protein in a biological sample from an individual, wherein the method comprises the following steps:
a) Obtaining a biological sample from an individual;
b) Analyzing the biological sample with the use of an immunoassay according to any one of claims 1 to 14, which is adapted to detect the biologically active protein.

By providing instructions in combination with the immunoassay, easy use of the kit as a PoCT can be ensured. This offers several technical advantages, including fast results due to shorter transportation time, immediate clinical decisions and improved patient management. PoCT minimizes possible pre-analytical errors, increases efficiency in emergency scenarios and supports timely treatment measures. The decentralized nature of PoCT facilitates the monitoring of chronic and infectious diseases.

In one embodiment, the use of the immunoassay as a lifestyle product, particularly for the detection of nutrition-associated parameters, is disclosed. This enables the tracking of parameters associated with malnutrition or undernutrition and is not a medical recommendation, but enables a person to recognize potential health risks. This can be used, for example, to independently monitor nutrition.

A lifestyle product for the purposes of this invention is an item or service that is provided because it conforms to a particular lifestyle, set of values or personal identity. These products often go beyond their functional purpose and are chosen by consumers to reflect and enhance their desired lifestyle, interests and self-expression, wherein a medical recommendation cannot necessarily be derived. In this embodiment, the immunoassay is not a medical product, but an aid with which a subject can check and optimize his or her lifestyle.

The present document further discloses a method for detecting a biologically active antigen in a biological sample, preferably urine, whole blood, saliva, milk or serum, comprising the following steps:
a) Obtaining a biological sample from an individual;
b) Analyzing the biological sample with the use of an immunoassay according to any one of claims 1 to 15 which is adapted to detect the biologically active antigen.

By this method, the use of the immunoassay according to the invention can be granted, whereby biologically active antigens can be analyzed quickly and accurately. In a particularly preferred embodiment, the biological sample is provided by the individual performing the immunoassay, in an alternatively embodiment the sample is obtained by a medically trained professional.

Preferably, no further treatment of the biological sample is carried out after collection. Particularly preferred, the biological sample can be applied directly to the immunoassay. This allows rapid and direct determination of a biologically active protein.

In one embodiment, the use of the immunoassay as point of care (PoC) diagnostics is disclosed, particularly for detecting antigens that are part of the epitope of a virus, preferably a pathogenic virus, e.g. influenza, SARS-COV-2, RSV, adenovirus, Strep A, norovirus, rotavirus, HIV, and/or comprising a recombinant antibody directed against human chorionic gonadotropin (hCG), preferably the recombinant antibody is an hCG antibody. It offers several technical advantages, including fast results due to shorter transportation time, immediate clinical decisions and improved patient management. PoCT minimizes possible pre-analytical errors, increases efficiency in emergency scenarios and supports timely treatment measures. The decentralized nature of PoCT facilitates the monitoring of chronic and infectious diseases.

In a preferably embodiment of the present invention, there is further disclosed at least one auxiliary protein for covering, masking and/or support protein in an immunoassay, wherein the auxiliary protein is recombinantly obtained from a stably transformed diatom or unicellular plant.

Immunoassays, e.g. ELISAs, dot blots, immunoblots and LFAs, require auxiliary proteins in addition to the antibodies, which can stabilize the antibody solutions on the one hand and serve to block the protein-binding surfaces on the other. For immunoassays, LFAs and dot blots, the surface is preferably nitrocellulose, for ELISAs preferably polystyrene. In the prior art, animal proteins such as bovine or calf serum or skimmed milk powder or casein are predominantly used. In order to be able to provide a vegan immunoassay in the sense of the present invention, preferably in a vegan LFA and/or vegan ELISA, these must be replaced by animal-free alternatives. Synthetically produced alternatives are known, e.g. ROTI®Block, but these are not suitable for all applications and are very cost-intensive and not economically competitive.

This document discloses, analogous to recombinant antibodies, the provision of auxiliary proteins derived from a stably transformed diatom or unicellular plant or higher plants. For this purpose, analogous to the antibodies, the sequence of bovine serum albumin (BSA) is codon-optimized and introduced as a gene into Phaeodactylum tricornutum and expressed there.

After digestion of the diatoms, the vegan BSA can be purified, preferably by affinity chromatography, or, in a preferably embodiment, used directly without further purification as a crude extract in various immunobiochemical methods.

Particularly preferably, the auxiliary protein according to the present invention is preferably a vegan protein expressed from a diatom, selected from the list including BSA, casein, or gelatin. Bovine serum albumin (BSA) is often used as a blocking agent to prevent non-specific binding of antibodies to the test surfaces and to reduce background noise. Casein is another blocking agent that helps to prevent non-specific interactions and improve the signal-to-noise ratio in assays, gelatin is a collagen-derived protein that can also be used to block non-specific binding in immunoassays.

In a preferably embodiment, the purification of the antibodies according to the invention follows a method known to the skilled person, i.e. after lysis and centrifugation and/or ultrafiltration, purification is carried out either via protein A, protein G or via the tag sequences used, for example the 6×His tag. Here lies another advantage of the method according to the invention, because in contrast to higher plants, which produce antibodies transiently or stably, diatoms do not contain any fibers that make the purification of the antibodies enormously more difficult. This enables a technically efficient and economically viable use of the antibodies obtained. The purification therefore largely corresponds to the method used, for example, for animal cell cultures such as CHO cells and is familiar to a person skilled in the art and is described below.

Preferably, the cells of the production clone, in this example a diatom, are destroyed by a so-called gentle disruption, wherein the product, the antibody in the sense of the present invention, is protected. Gentle methods include, for example, high pressure, electrical voltage, ultrasound or disruption by collision in so-called vibrating mills. In a particularly preferred embodiment, the separation is carried out by a combination of Manton-Gaulin homogenizer and subsequent ultrasonic treatment with Covaris E220 Focused Ultrasonicator.

After digestion, the so-called lysate is available, which contains the entire cell content. In order to obtain a functional antibody, further purification steps are preferably carried out. Non-soluble components are separated from the soluble components, e.g. by centrifugation or filtration, particularly preferred by a centrifugation sequence. Pigment-containing components are separated, for example, by filtration, cooling, chemical precipitation, ion exchange chromatography or size exchange chromatography. Preferably, a sequence of filtration, cooling, chemical precipitation, ion exchange chromatography or size exchange chromatography is used to separate components sequentially and efficiently.

The antibodies are purified by so-called affinity chromatography. This can be done using typical purification methods for antibodies such as protein A or G or using so-called affinity tags. In addition, impurities are removed by tangential flow filtration or dead-end filtration and/or dialysis. Finally, the antibody is transferred to a suitable buffer solution.

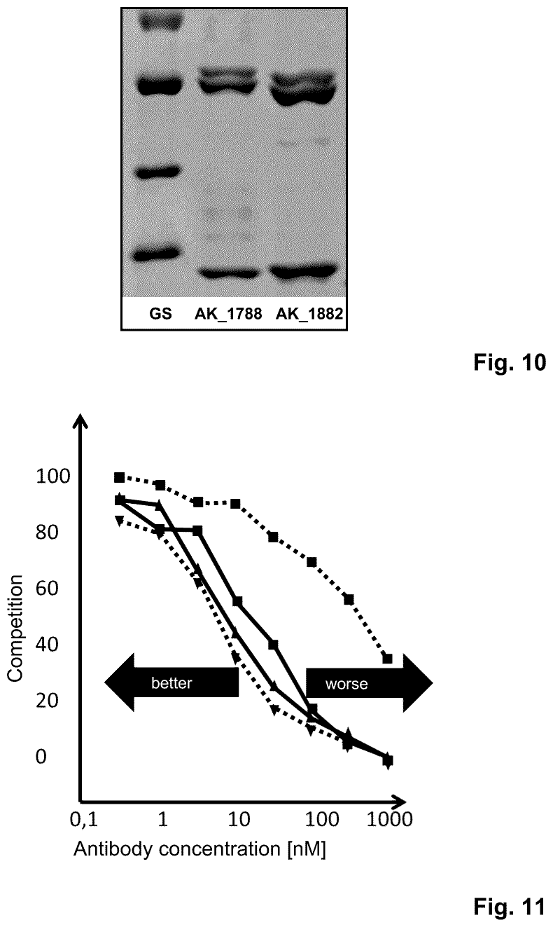

In order to achieve good performance, the antibodies produced must have a certain degree of purity. The purity of the antibodies produced can be checked by so-called polyacrylamide gel electrophoresis. FIG. 10 shows an example of two different anti-hCG antibodies according to the invention, here labeled AK_1788 and AK_1882, which have a high degree of purity.

Gel electrophoresis is a laboratory technique that can be used to separate and analyze molecules such as DNA, RNA and proteins based on their size and charge. The molecules are placed in a gel matrix and an electric field is applied, which causes the molecules to migrate through the gel. Smaller molecules move faster and migrate further, resulting in distinct bands or patterns that can be visualized. The technical advantages of gel electrophoresis include its ability to separate complex mixtures of molecules with high resolution. It is versatile, can accommodate different types of molecules and provides qualitative and semi-quantitative information about their properties.

A dot blot is a laboratory technique used in molecular biology and immunology to detect, analyze and quantify specific biomolecules such as proteins or nucleic acids (DNA or RNA) in a sample. In a dot blot, a small amount of the target biomolecule is immobilized or “blotted” onto a solid support, usually a membrane. This immobilization can be done by directly dabbing the sample onto the membrane.

### REFERENCE LIST

1 Immunoassay/device
2 Sample application area
3 Capture area
4 Conjugate area
5 Control zone
6 Membrane
7 Fixation area
8 Housing
9 Embossed seam
10 Breaking point
11 Sequence optimization of the nucleic acid sequence
12 Insertion of the nucleic acid sequence into a vector
13 Transformation
14 Screening method
Method for the production of recombinant proteins
16 Vector
17 Cells
18 Cell culture

### DESIGN EXAMPLES

The present invention is explained in more detail with reference to the following figures and embodiments, without limiting the invention to these.

This shows

FIG. 1: an immunoassay, particularly a lateral flow immunoassay (test strip) in a schematic view from above;

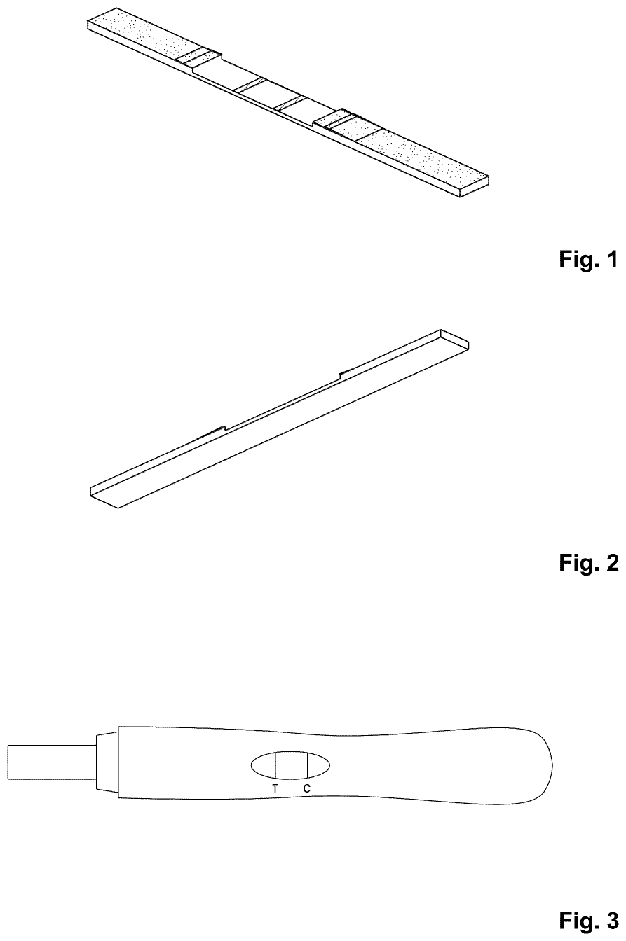

FIG. 2: a schematic view from below of an immunoassay, particularly a lateral flow immunoassay (test strip);

FIG. 3: a schematic view of a device in which an immunoassay, particularly a lateral flow immunoassay (test strip), is arranged inside a housing;

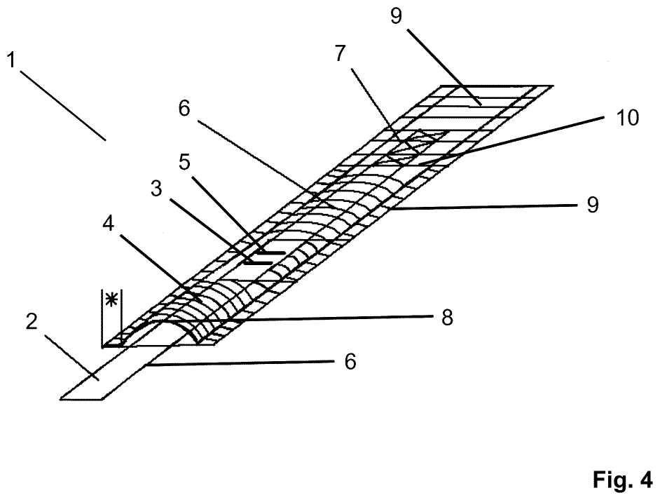

FIG. 4: the schematic view of a device in which the housing is formed from two layers of paper or paperboard arranged one above the other;

FIG. 5: Glycan analysis to determine the composition of the glycosylation of an IgG (antibody) in a hamster cell culture;

FIG. 6: Glycan analysis to determine the composition of the glycosylation of the IgG (antibody) from FIG. 5 in Expi cells (human cell culture);

FIG. 7: Glycan analysis to determine the composition of the glycosylation of the IgG (antibody) from FIGS. 5 and 6 in Phaeodactylum.

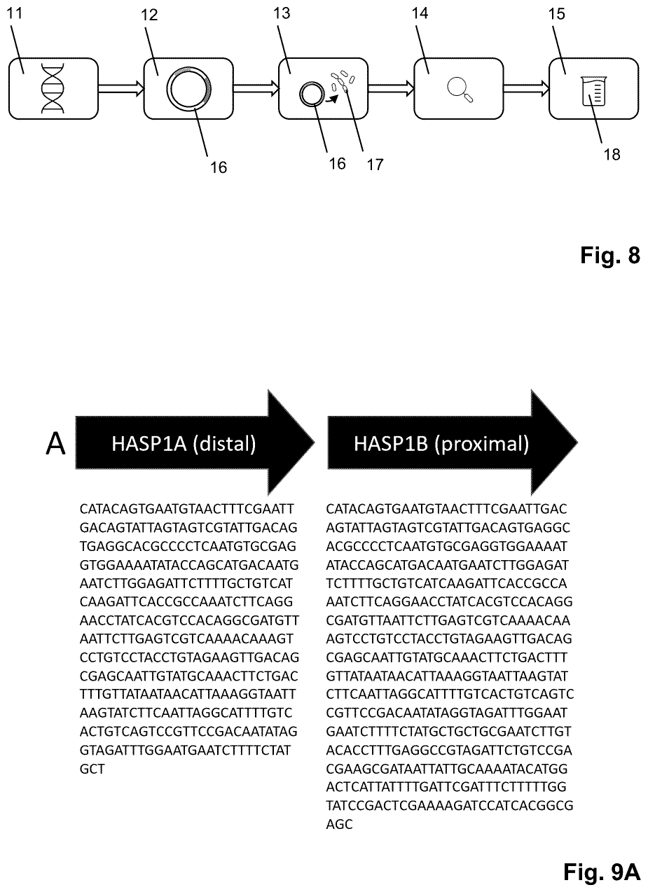

FIG. 8: A schematic overview of the provision of antibodies according to the invention.

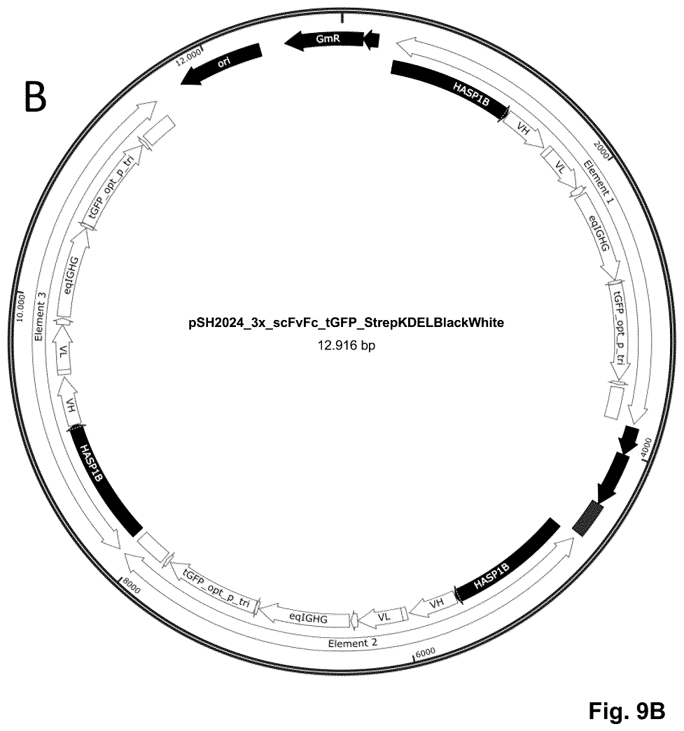

FIG. 9: the repetition of two promoters

FIG. 10: Polyacrylamide gel electrophoresis with anti-hCG antibody; GS=size standard, AK_1788 and AK_1882: anti-hCG antibody, stained with Coomassie.

FIG. 11: A diagram comparing the binding affinity of two diatom antibody (formats) with the sequence matches from human cell culture (Expi293F)

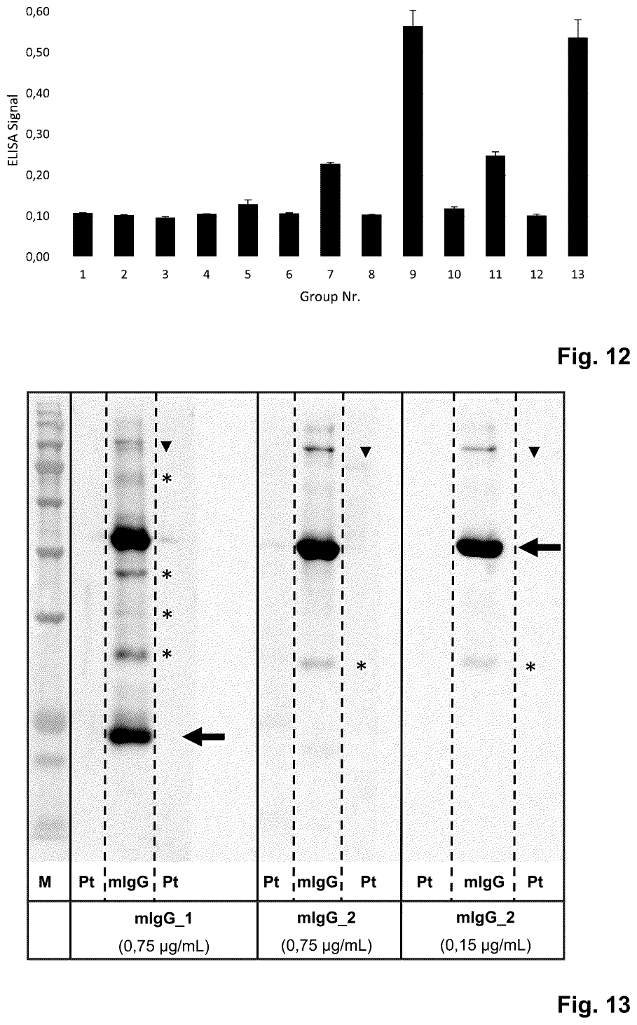

FIG. 12: ELISA for the detection of human beta-chorionic gonadotropin (hCG) by antibodies produced in diatoms

FIG. 13: Use of antibodies from diatoms according to the invention as secondary antibodies labeled with horseradish peroxidase for the detection of primary antibodies in an immunoblot (SDS page). mIgG: monoclonal IgG against human interleukin 15 from mouse; mIgG_1: an affinity-purified polyclonal antibody produced in goat (Goat-α-mouse-IgG-HrRP), directed against the light and heavy chain of mouse IgG (comparative antibody); mIgG_2: antibody according to the invention from P. tricornutum, directed against the heavy chain of mouse IgG (antibody according to the invention for immunoassay)

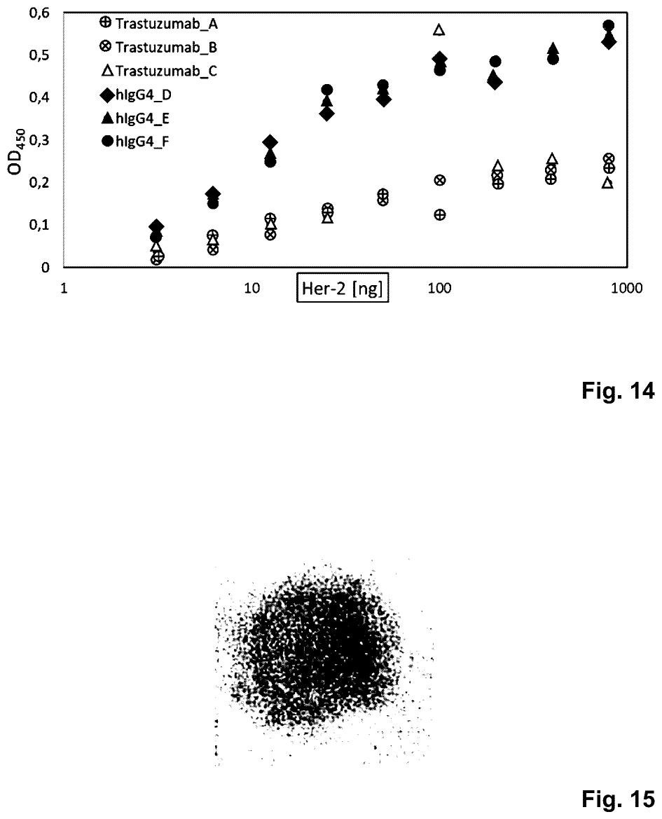

FIG. 14: ELISA against the Herceptin 2 receptor (Her2), performed with a trastuzumab biosimilar from rabbits (trastuzumab A-C) and with a biosimilar from the diatom P. tricornutum (hIgG4_D-F).

FIG. 15: Dot blot with anti-hCG antibodies AK_1788 and AK_1882

FIG. 8 shows a schematic overview of the context of the provision of a diatom for the production of the recombinant antibodies of the immunoassay according to the invention. All the sub-steps shown (individually or in combination) lead to an improvement/optimization of the heterologous production of proteins, particularly of antibodies, especially in the diatom Phaeodactylum tricornutum.

Firstly, a sequence optimization (11) of a nucleic acid sequence is carried out. This may include, for example, codon optimization and/or the use of particularly protease-resistant genetic elements (as described herein), such as hinge regions derived from equine IgGs (immunoglobulin G).

The nucleic acid sequence is then introduced into a vector (16) (or an isolated nucleic acid), wherein individual genetic elements and/or complete expression cassettes are used repetitively. Furthermore, special inducible promoters are used, particularly a promoter element from the nucleic acid sequence of SEQ ID NO: 1.

By utilizing specific signal sequences (as described herein), expression of the proteins to be heterologously produced occurs in the endoplasmic reticulum of the cells, resulting in protection of the proteins from proteases and successful glycosylation, thereby increasing yield and maintaining the functionality of the proteins.

In a next step, the vector (or nucleic acid) is transformed (13) into target cells, preferably into photosynthetically active cells, particularly cells of a unicellular plant or Viridiplantae, preferably cells of Phaeodactylum tricornutum. The transformation can be carried out ballistically or by means of electroporation in a suitable medium.

This is followed by a screening method (14) to select cells with an increased expression rate of a nucleic acid sequence. Screening can be carried out using reporter genes for high-performance producers, i.e. for cells with an increased expression rate. Furthermore, the correlation between the expression of the reporter genes and the expression level of the proteins to be produced can be determined. Preferably, multiwell plates are used both for the screening method and for culture monitoring.

Following the screening process, a method for producing recombinant proteins, preferably recombinant antibodies, can be carried out on the basis of the cells with an increased expression rate determined in the screening process.

Recombinant proteins (15) are produced in a culture medium that is adapted to the genetic elements used, particularly the promoters used. Even the cultivation conditions, such as minimum light intensities and aeration quantities, are adapted.

Both in terms of nucleic acid sequence and in terms of the vector or isolated nucleic acid, the use of repetitive genetic elements leads to an enormous increase in expression rates in P. tricornutum. FIG. 9A shows the repetition of two HASP1mod promoters. FIG. 9B shows an example of an embodiment of a vector according to the invention in which an expression cassette has been used repetitively, here in the form of a triple cassette for the production of antibodies in scFv-Fc format. In particular, this repetitive use of the expression cassette leads to a significant increase in the yield as well as the number of clones or the proportion of clones that show detectable production, which minimizes the overall process effort. In the embodiment example shown, the expression cassette has a promoter element (9.1), a first transcription unit which codes for a protein to be produced recombinantly, and a second transcription unit which codes for the reporter gene gfp, wherein the individual genetic elements are applied to one expression cassette by way of example, but are also present in the other expression cassettes.

FIG. 12 shows an ELISA according to the invention for the detection of human beta-chorionic gonadotropin (hCG) by antibodies produced in diatoms. In FIG. 12, the ELISA was performed according to this method:

After coupling human chorionic gonadotropin (hCG) to the ELISA plate, the first antibody directed against hCG was added after saturation with an animal product-free blocking reagent, which was produced in diatoms and purified by affinity chromatography.

After the washing step, a second diatom-based antibody was used for amplification and an antibody coupled with the enzyme HRP for signal generation to start the detection reaction after addition of the substrate.

In this ELISA, a total of 13 test series were prepared, of which two samples should show a strong signal and eleven serve as a control in order to obtain a meaningful result. The corresponding approaches are listed in Table 1, wherein the order from left to right corresponds to the work steps. The number of steps may vary. In this case, 5 steps were carried out. First, the antigen, in this case hCG, was bound to the plate. Free binding sites were then filled using a so-called block solution. From the 3rd step onwards, the additions differed, see Table 1. These different additions enable the characterization of the antibodies and serve, among other things, to exclude unspecific binding.

A total of three different antibodies according to the invention, which are produced in Phaeodactylum tricornutum, were used in this exemplary ELISA:

On the one hand, two different antibodies according to the invention were used, which are directed against the beta subunit of hCG and correspond to a human IgG4 (AK_1788 and AK_1892). On the other hand, a secondary antibody according to the invention obtained from diatoms was used, which corresponds to a mouse antibody directed against human IgG4 (AK_2073). In this example, detection was performed with a polyclonal antibody directed against mouse IgG and conjugated to a horseradish peroxidase (HRP) (G-α-mIgG-HRP).

Table 1 lists the antibody additions coupled with the image. A signal is only expected in samples 9 and 13, all other samples are for control purposes.

TABLE 1
Groups for ELISA with antigens obtained from diatoms. anti-mIgG-HRP =
Antibody labeled with HRP directed against antibody 2073 (AK_2073).
AK_1788 and AK_1882 are antibodies directed against hCG,
AK_2073 is an antibody directed against antibodies 1788 and 1882.

| Group | Coating | 1. Addition | 2. Addition | 3. Addition | 4. Addition |
|---|---|---|---|---|---|
| 1 | Block solution | Block solution | Block solution | Block solution | |
| 2 | hCG | Block solution | Block solution | Block solution | |
| 3 | hCG | Block solution | Block solution | Block solution | anti-mIgG- |
| | | | | | HRP |
| 4 | hCG | Block solution | Block solution | AK_2073 | |
| 5 | hCG | Block solution | Block solution | AK_2073 | anti-mIgG- |
| | | | | | HRP |
| 6 | hCG | Block solution | AK_1788 | Block solution | |
| 7 | hCG | Block solution | AK_1788 | Block solution | anti-mIgG- |
| | | | | | HRP |
| 8 | hCG | Block solution | AK_1788 | AK_2073 | |
| 9 | hCG | Block solution | AK_1788 | AK_2073 | anti-mIgG- |
| | | | | | HRP |
| 10 | hCG | Block solution | AK_1882 | Block solution | |
| 11 | hCG | Block solution | AK_1882 | Block solution | anti-mIgG- |
| | | | | | HRP |
| 12 | hCG | Block solution | AK_1882 | AK_2073 | |
| 13 | hCG | Block solution | AK_1882 | AK_2073 | anti-mIgG- |
| | | | | | HRP |

It can be seen from FIG. 12 that only the groups 9 and 13 highlighted in Table 1 have a strong signal with an intensity of 0.57 and 0.54 respectively. This shows that the first and second antibodies have a high affinity for the antigen and the first antibody, respectively. By adding G-α-mIgG-HRP as a detection antibody, it was shown that these antibodies are selective for the detection of human beta-chorionic gonadotropin (hCG).

Here, all other control groups do not show a value of over 0.25. This allows the conclusion that both the AK_1788 and AK_1892 antibodies selectively bind the antigen, in this case hCG.

For the so-called control antibody 2073 required in the LFA, which is placed on the control line, it can be shown that it can bind the antibodies AK_1788 and AK_1892 as desired.

Thus, the antibodies shown here are complete and functional for the construction of an LFA, in this case a pregnancy test, and enable the detection of pregnancy as well as the staining of the control line of an LFA.

FIG. 13 shows an exemplary use of an antibody according to the invention in an immunoassay (immunoblot) in the SDS PAGE variant. Here, the diatom antibodies according to the invention are used as secondary antibodies labeled with horseradish peroxidase for detecting primary antibodies. In FIG. 13, mIgG stands for a monoclonal IgG against human interleukin 15 from mice, and Pt is the abbreviation for a protein extract from the diatom Phaeodactylum tricornutum (Pt), which is used as a negative control to show that the antibodies do not recognize proteins from the diatoms. Both mIgG and Pt were separated by SDS-PAGE, the separated proteins from the gel were then transferred to a membrane by Western blot and subsequently the mIgG was detected with a commercial reference antibody (mIgG_1) and two different concentrations of an antibody according to the invention.

The comparator antibody used is mIgG_1, a commercial, polyclonal, affinity-purified antibody (Goat-α-mouse-IgG-HrRP), which is produced in a goat, directed against the light and heavy chain of mouse IgG and coupled with horseradish peroxidase (HRP) (comparator antibody). The antibody mIgG_2 (Pt-α-mouse IgG-HRP) according to the invention is used in two concentrations (0.75 μg/mL and 0.15 μg/mL). This was obtained from the diatom Phaeodactylum tricornutum and is directed against the heavy chain of mouse IgG and coupled with horseradish peroxidase (HRP). BlueStar from Nippon Genetics was used as the length standard (M).

The antibody Goat-α-mouse-IgG-HRP produced in goats recognizes both the light and the heavy chain of mouse mIgG, which is why two strong specific signals can be seen in addition to numerous non-specific signals (arrows). The antibody from diatoms (mIgG_2) is only directed against the heavy chain (HC, upper arrow only), which is why the light chain, which produces the lower specific band in the reference antibody, is not displayed. The antibody from diatoms (mIgG_2) is only directed against the heavy chain (HC, upper arrow only), which is why the lower band is not recognized. The antibody from diatoms provides similarly clear and, above all, more specific signals even in small quantities. Several non-specific bands (*) appear, especially with the commercial animal antibody (mIgG_1). The uppermost bands are still complete IgGs (▾), in which the denaturation characteristic of SDS-PAGE has not led to a separation of the IgG into the light and heavy chains. Thus, the antibodies according to the invention not only show a strong signal amplification at a lower concentration, but also a higher specificity compared to the target antibodies, which is why they represent a more specific alternative to animal antibodies. This increased sensitivity enables a lower quantity of antibodies to be used, while the higher specificity allows a more reliable determination and the avoidance of false-positive results. It was also shown that the antigens have no affinity for the diatom-specific proteins, as demonstrated by the lack of bands in the separated protein extracts from P. tricornutum (Pt).

FIG. 14 shows an ELISA of animal antibodies and antibodies according to the invention against the Herceptin 2 receptor (Her2), carried out with a trastuzumab biosimilar from rabbits (trastuzumab A-C) and with a biosimilar from the diatom Phaeodactylum tricornutum (hIgG4_D-F).

The OD value refers to the value of the optical density. It is a measure of the absorption of light by a sample in a microplate well. The OD value is used to quantify the presence or concentration of a particular molecule, in this case an antigen, in the tested sample. Higher OD values generally indicate a higher concentration of the target molecule in the sample, while lower values indicate lower concentrations. This measurement is a critical component in evaluating the results of ELISA experiments and determining the strength of the reaction between antigen and antibody.

The OD values in this example show that across all concentration ranges the antibodies obtained from the diatom Phaeodactylum tricornutum (hIgG4_D-F) show a higher specific activity against the antigen Herceptin 2 receptor than the animal analogs (Trastuzumab A-C). This is probably due to a higher purity of the antibodies according to the invention, which is also demonstrated in FIG. 5-7 (glycosylation pattern), FIG. 13 (SDS page immunoblot), among others. The higher activity enables a more sensitive detection of the antigens and thus a lowering of the detection limit. Due to the low concentration, corresponding antigens can, for example, be detected earlier in the course of the disease and thus contribute to rapid detection and treatment, which can, for example, interrupt chains of infection.

FIG. 15 shows a dot blot assay. In the dot blot assay, one of the antibodies according to the invention (AK_1788, directed against hCG) is conjugated with colloidal gold and another antibody according to the invention (AK_1882, directed against another epitope on hCG) is applied in a circle to a suitable membrane. The membrane and the antibodies labeled with colloidal gold are then incubated together with a solution containing hCG. The anti-hCG antibodies used from the ELISA (FIG. 13) are therefore tested in a set-up that corresponds to an LFA model. Both the membrane-bound antibodies and the antibodies labeled with colloidal gold simultaneously bind the hCG from the solution. If both antibodies are able to bind the hCG, there is a clear discoloration at the position of the membrane-bound antibody compared to the surrounding area. FIG. 15 shows this staining with the two anti-hCG antibodies. This shows that both antibodies bind simultaneously to the hCG molecule and can therefore detect hCG using a typical LFA setup.

### Example 1: Production of Antibodies from Diatoms and Purification for an Immunoassay

The embodiment example, the procedure of which is outlined in FIG. 8, shows the production of an antibody using the method according to the invention. This antibody in IgG format is directed against equine interleukin 31 and was produced with more than 160 mg of purified eqIgG*L⁻¹ cell culture within a 14-day culture period.

In a first step, the codon usage is adapted to P. tricornutum. In the present case, the starting sequence originates from a human scFv bank and was codon-optimized before use in the diatoms. The required genetic elements, typically the variable region of the light chain (VL) and the variable region of the heavy chain (VH), were synthesized by the company IDT-DNA (Coralville, lowa) so that they fit optimally into the vectors created (FIG. 9B). Dabei wurden auch störende Restriktionsstellen entfernt, bzw. modifiziert. Interfering restriction sites were also removed or modified. A variant of the vectors according to the invention for the production of antibodies in scFv-Fc format is shown in FIG. 9B. Here, the variable region of the light chain (kappa or lambda format (VLK oder VLA)) and the variable region of the heavy chain (VH) can be used. In this example, the constant regions of both chains originate from the horse. Constructs containing constant antibody regions from other host organisms (e.g. mouse or human) have also been generated. Further variants of the vectors according to the invention have been produced and successfully used for the heterologous production of other formats such as Fab or scFv-Fc. The example shown in FIG. 9B contains the finished vector construct and, in addition to the elements to be introduced into P. tricornutum, bacterial genetic elements used for cloning in E. coli (colE1 origin and gentamycin resistance gene). Three copies of the gene of interest were used in the construct. After the ballistic or electroporation-based transformation of P. tricornutum, the clones obtained must not only be checked for the uptake of the antibody genes, but also the rapid identification of clones that express the introduced antibody gene particularly strongly. The underlying screening method according to the invention makes it possible to correlate the fluorescence caused by gfp (Green Fluorescent Protein), which is measured in a special microtiter plate reader, with the amount of antibody expected to be formed later.

Compared to production rates of a maximum of 3 mg 3 mg antibody*L⁻¹ culture described in the prior art, 160 mg purified antibody could be obtained from 1 L culture in the system according to the invention. To achieve this, new media compositions, aeration with more than 3 L*min⁻¹ of compressed air and lighting with a light intensity of 100-1000 W*m⁻² were used in a reactor column.

An exemplary media composition is shown in Table 2:

TABLE 2
Media composition

| Substance | Concentration |
|---|---|
| Tris-HCl pH 8 | 10 mM |
| NaNO₃ | 30 mM |
| vitamins for P. tricornutum | over 10x (f/2 Medium) |
| trace elements for P. tricornutum | over 10x (f/2 Medium) |
| Glycerin | at least 100 mM |
| NH₂PO₄ | at least 360 μM |

After typically fourteen to twenty days of fed-batch culture, the diatoms can be harvested and digested.

The purification follows the classical method, i.e. after lysis and centrifugation and/or ultrafiltration, purification is carried out either via protein A, protein G or via the tag sequences used, for example the 6×His tag. This is another advantage of the immunoassay according to the invention, because in contrast to higher plants, which produce antibodies transiently or stably, diatoms do not contain any fibers that make purification of the antibodies enormously more difficult. The purification therefore largely corresponds to the method used, for example, for animal cell cultures such as CHO cells and is familiar to a person skilled in the art and is described below.

The cells of the production clone, in this example a diatom, are destroyed by a so-called gentle disruption, wherein the product, the antibody in the sense of the present invention, is protected. Gentle methods include, for example, high pressure, electrical voltage, ultrasound or disruption by collision in so-called vibrating mills; in this case, a combination of Manton-Gaulin homogenizer and subsequent ultrasound treatment in the Ultrasonicator was carried out. After digestion, the so-called lysate is available, which contains the entire cell content. In order to obtain a functional antibody, purification steps are carried out. Non-soluble components are separated from the soluble components, e.g. by centrifugation or filtration, in this case a centrifugation sequence. Pigment-containing components are separated by a sequence of filtration, cooling, chemical precipitation, ion exchange chromatography or size exchange chromatography.

The antibodies are purified by affinity chromatography. This is carried out either using a typical purification method for antibodies, such as protein A or G, or using so-called affinity tags. In addition, impurities are removed by tangential flow filtration or dead-end filtration and/or dialysis. Finally, the antibody is transferred to a suitable buffer solution.

In order to achieve good performance, the antibodies produced must have a certain degree of purity. In this example, the purity of the antibodies produced is checked by so-called polyacrylamide gel electrophoresis. FIG. 10 shows an example of two different anti-hCG antibodies according to the invention, designated here as A-hCG-1 and A-hCG-12, which have a high degree of purity.

### Example 2: Provision of a Pregnancy Test

The lateral flow pregnancy test uses intracellularly produced antibodies from the diatom Phaeodactylum tricornutum. These antibodies have a concentration of about 160-360 mg/L per liter of culture when produced. These antibodies are specifically targeted to human chorionic gonadotropin (hCG), consisting of an α-subunit of 92 amino acids and a β-subunit of 145 amino acids. The test comprises a first antibody, a capture antibody, a second antibody, a detection antibody and another antibody, a control antibody.

The detection antibodies are characterized by the fact that they are labeled with colloidal gold nanoparticles. The first immobilized antibody, which is located in the capture zone, acts as the capture antibody. The second antibody, which is located in the conjugation zone and is referred to as the detection antibody, is also directed against the antigen, preferably against a different epitope of the antigen than the detection antibody. This second antibody is conjugated to a marker particle, in this case colloidal gold nanoparticles.

In a preferably embodiment of the immunoassay, the first antibody and/or the second antibody, in particular the mobilized second antibody, is labeled with a dye and/or an optically active nanoparticle, in particular a gold nanoparticle.

Ein weiterer Antikörper, der sich vorzugsweise in der Kontrollregion (Kontrollzone) befindet und immobilisiert ist, richtet sich gegen einen Detektionsantikörper.

The purified antibodies, including the first, second and subsequent antibody, are combined with a plant-derived cover protein (diatom-expressed BSA) and applied to a nitrocellulose membrane. This configuration creates a lateral flow immunoassay that has a sample application area, a conjugate area, and a capture area integrated on the membrane. Sample application area and a capture area are fluid-connected to each other on the membrane via a flow path, wherein the conjugate area is located in the flow path. This design facilitates the performance of the test and the interpretation of the results.

This membrane is surrounded by a housing within the meaning of the present invention, consisting predominantly (at least 90%) of cellulose fibers, which on the one hand is water-resistant (at least for transport and service life) and on the other hand is made from sustainable, preferably recovered plant fibers and is biodegradable (e.g. by composting).

For easy use as a PoCT, markings (symbols, characters, geometric shapes) are attached to the housing on the membrane, which allow easy interpretation of the immunoassay result. Particularly in the embodiment of a kit in which instructions are provided in addition to the assay according to the invention, ease of use is also ensured for non-medical personnel.

1-27. (canceled)

28. An immunoassay for detecting a biologically active antigen, particularly a hormone, a protein or a pharmaceutical substance, in a biological sample from an individual, provided with:
a sample application area for applying the biological sample from the individual, wherein the biological sample is preferably urine, whole blood, saliva, milk or serum;
a capture area, wherein the capture area is provided with an immobilized first antibody that is directed against the biologically active antigen, in particular against the hormone, the protein, the peptide or the pharmaceutical substance;
a conjugate area, wherein the conjugate area is provided with a second antibody directed against the biologically active antigen, particularly against the hormone, protein, peptide or pharmaceutical substance;
characterized in that
the first antibody and the second antibody are a recombinant antibody obtained from a diatom or unicellular plant,
wherein the immunoassay is provided with at least one further antibody and/or one auxiliary protein,
wherein the auxiliary protein, as a blocker, saturates free surfaces of a reaction vessel or a membrane,
wherein the other antibody and the auxiliary protein are obtained from the diatom and/or a Viridiplantae and/or the unicellular plant, and
wherein the recombinant antibody is provided in a purity of at least 90%, and
wherein the immunoassay is vegan.

29. The immunoassay according to claim 28, wherein the recombinant antibody is obtained in a concentration of 20-1000 mg/L, preferably 30-800 mg/L, alternatively at least 30-160 mg/L of a culture of a diatom.

30. The immunoassay according to claim 28, wherein the glycosylation of the first antibody and/or the second antibody has a modified glycosylation pattern compared to the corresponding native antibody, preferably the glycosylation has a more homogeneous pattern with a homogeneity factor in the range of 1 to 3, particularly preferred the glycosylation has a homogeneous mannose-rich N-glycan pattern with a homogeneity factor in the range, preferably in the range of 1 to 3.

31. The immunoassay according to claim 28, wherein the recombinant antibody is a mosaic antibody, wherein the mosaic antibody comprises at least one first sequence selected from at least a first organism and at least one second sequence selected from at least a second organism.

32. The immunoassay according to claim 28, wherein the amino acid sequence of the first antibody and/or the second antibody
a) comprises a vertebral, preferably mammalian, particularly preferred human, antibody; or
b) comprises or consists of an amino acid sequence which has at least 80%, preferably at least 85%, particularly preferred at least 90%, most particularly preferred at least 95%, in particular at least 97% sequence identity to homologous sequence regions of a vertebral and/or mammalian and/or human antibody.

33. The immunoassay according to claim 28, wherein the nucleic acid sequence encoding the first antibody and/or the second antibody is codon optimized for the host organism from which the first antibody and/or the second antibody is derived.

34. The immunoassay according to claim 28, wherein the amino acid sequence of the recombinant antibody in the hinge region is modified in such a way that it has an increased stability, preferably with a stability factor of 1.1 to 5, with respect to diatom-, plant- or microalga-specific proteases compared to the native antibody.

35. The immunoassay according to claim 28, wherein the recombinant antibody is expressed from a stably transformed diatom or unicellular plant, preferably from a stably transformed diatom, for several generations, preferably for at least 60 generations, more preferably for at least 80 generations, most preferably for at least 100 generations.

36. The immunoassay according to claim 28, wherein the recombinant antibody is expressed intracellularly, preferably in a stably transformed diatom.

37. The immunoassay according to claim 28, wherein the first antibody and/or the second antibody is an antibody directed against human chorionic gonadotropin (hCG).

38. The immunoassay according to claim 28, wherein the biologically active antigen is a part of the epitope of a virus, preferably a pathogenic virus, for example influenza, SARS-COV-2, RSV, adenovirus, Strep A, norovirus, rotavirus, HIV.

39. The immunoassay according to claim 28, wherein the biologically active antigen is a tumor-associated sequence, preferably an HLA complex and/or a sequenced part of a tumor epitope and/or a tumor marker, for example IFN-γ, IL-8, PSA, CEA, AFP, DCP, CA 125, HER2/neu.

40. The immunoassay according to claim 28, wherein the biologically active antigen is a characteristic sequence for identifying a protein, preferably an enzyme tag, particularly preferred selected from the list consisting of His6 tag, Strep tag, c-Myk tag, Flag tag and GST tag.

41. The immunoassay according to claim 28, wherein the biologically active antigen is a sequence associated with nutritional parameters, for example transcobalamin II, ferritin, homocysteine, eicosapentaenoic acid (EPA) and docosahexaenoic acid (DHA), or calcitriol.

42. The immunoassay according to claim 28, wherein the diatom is Phaeodactylum tricornutum.

43. The immunoassay according to claim 28, wherein the immunoassay is a lateral flow immunoassay providing fluid-connected at least one sample application area, a conjugate area and a capture area fluid-connected on a membrane.

44. The immunoassay according to claim 28, wherein the immunoassay is carried out as an enzyme-linked immunosorbent assay (ELISA) immunoassay, wherein at least
a) a sample application area for applying a biological sample is provided, which is
b) confined by a capture area which is designed as a vessel boundary and/or part of a vessel boundary, preferably as a microtiter plate, and
c) a conjugate area, which is spatially arranged in the sample application area.

45. The immunoassay according to claim 44, wherein the second antibody is provided as an antibody-enzyme conjugate.

46. A device, provided with:
a container, particularly a housing, and
an immunoassay according to claim 28 arranged therein, particularly a lateral flow immunoassay.

47. A method for detecting a biologically active protein in a biological sample, preferably urine, whole blood, saliva, milk or serum, comprising the following steps:
a) Obtaining the biological sample from the individual;
b) Analyzing the biological sample with the use of an immunoassay according to claim 28, which is adapted to detect the biologically active protein.


SEQUENCE LISTING (5 sequences)

SEQ ID NO: 1 - DNA, 864 bp, synthetic construct
  source 1..864: mol_type = other DNA
catacagtgaatgtaactttcgaattgacagtattagtagtcgtattgacagtgaggcacgcccctcaatgtgcgaggtggaaaatataccagcatgacaatgaatcttggagattcttttgctgtcatcaagattcaccgccaaatcttcaggaacctatcacgtccacaggcgatgttaattcttgagtcgtcaaaacaaagtcctgtcctacctgtagaagttgacagcgagcaattgtatgcaaacttctgactttgttataataacattaaaggtaattaagtatcttcaattaggcattttgtcactgtcagtccgttccgacaatataggtagatttggaatgaatcttttctatgctcatacagtgaatgtaactttcgaattgacagtattagtagtcgtattgacagtgaggcacgcccctcaatgtgcgaggtggaaaatataccagcatgacaatgaatcttggagattcttttgctgtcatcaagattcaccgccaaatcttcaggaacctatcacgtccacaggcgatgttaattcttgagtcgtcaaaacaaagtcctgtcctacctgtagaagttgacagcgagcaattgtatgcaaacttctgactttgttataataacattaaaggtaattaagtatcttcaattaggcattttgtcactgtcagtccgttccgacaatataggtagatttggaatgaatcttttctatgctgctgcgaatcttgtacacctttgaggccgtagattctgtccgacgaagcgataattattgcaaaatacatggactcattattttgattcgatttctttttggtatccgactcgaaaagatccatcacggcgagc

SEQ ID NO: 2 - protein, 12 aa, synthetic construct
VIKEPCCCPKCP

SEQ ID NO: 3 - protein, 10 aa, synthetic construct
EQKLISEEDL

SEQ ID NO: 4 - protein, 8 aa, synthetic construct
WSHPQFEK

SEQ ID NO: 5 - protein, 8 aa, synthetic construct
DYKDDDDK